(19) United States
(12) Patent Application Publication
(10) Pub. No.: US 2001/0049108 A1
(43) Pub. Date: Dec. 6, 2001

(54) Methods for reducing non-specific binding to an oligonucleotide array

(76) Inventors: Glenn McGall, Mountain View, CA (US); Martin Goldberg, San Jose, CA (US); Thomas B. Ryder, Los Gatos, CA (US); Steve Woodman, San Jose, CA (US)

Correspondence Address: TOWNSEND AND TOWNSEND AND CREW, TWO EMBARCADERO CENTER, EIGHTH FLOOR, SAN FRANCISCO, CA, 94111-3834, US

(21) Appl. No.: 09/862,571

(22) Filed: May 23, 2001

Related U.S. Application Data
(63) Continuation of application No. 09/063,311, filed on Apr. 20, 1998.

(51) Int. Cl. C12Q 1/68; C07H 21/02; C07H 21/04; C12P 19/34

(52) U.S. Cl. 435/6; 435/91.1; 536/25.32

(57) ABSTRACT

The present invention provides a variety of methods for reducing non-specific binding of a target molecule or plurality of target molecules to an array of oligonucleotides. The methods of the present invention include surface modification techniques and oligonucleotide modification techniques. According to one method of the present invention, non-specific binding of a target molecule to an array of oligonucleotides is reduced by replacing at least one of: i) the protecting groups on each of the plurality of oligonucleotides, and ii) the protecting groups on each of the protected regions of the substrate, with a negatively charged phosphate residue.

### BACKGROUND OF THE INVENTION

The present invention relates to the field of solid phase polymer synthesis. More specifically, the invention provides methods and supports which find application in solid phase synthesis of oligonucleotide arrays or of single compounds on a preparative scale. The oligonucleotide arrays may be used, for example, in screening studies for determination of binding affinity and in diagnostic applications. The surface modifications of the present invention reduce non-specific binding of target molecules to the arrays in the binding affinity and diagnostic applications.

The synthesis of biological polymers such as peptides and oligonucleotides has been evolving in dramatic fashion from the earliest stages of solution synthesis to solid phase synthesis of a single polymer to the more recent preparations of libraries having large numbers of diverse oligonucleotide sequences on a single solid support or chip.

Improved methods of forming large arrays of oligonucleotides, peptides and other polymer sequences in a short period of time have been devised. Of particular note, Pirrung et al., U.S. Pat. No. 5,143,854 (see also PCT Application No. WO 90/15070) and Fodor et al., PCT Publication No. WO 92/10092, all incorporated herein by reference, disclose methods of forming vast arrays of peptides, oligonucleotides and other polymer sequences using, for example, light-directed synthesis techniques. See also, Fodor et al., Science, 251:767-777 (1991), also incorporated herein by reference for all purposes. These procedures are now referred to as VLSIPS™ procedures.

In the above-referenced Fodor et al., PCT application, an elegant method is described for using a computer-controlled system to direct a VLSIPS™ procedure. Using this approach, one heterogenous array of polymers is converted, through simultaneous coupling at a number of reaction sites, into a different heterogenous array. See, U.S. Pat. Nos. 5,384,261 and 5,677,195 to Winkler et al., the disclosures of which are incorporated herein for all purposes.

The development of VLSIPS™ technology as described in the above-noted U.S. Pat. No. 5,143,854 and PCT Publication Nos. WO 90/15070 and 92/10092, is considered pioneering technology in the fields of combinatorial synthesis and screening of combinatorial libraries. More recently, U.S. patent application Ser. No. 08/082,937, filed Jun. 25, 1993, describes methods for making arrays of oligonucleotide probes that can be used to provide a partial or complete sequence of a target nucleic acid and to detect the presence of a nucleic acid containing a specific oligonucleotide sequence.

The control of surface properties to optimize VLSIPS™ substrate performance in both chemical synthesis and bioassays has been recognized to involve such parameters as site density for synthesis initiation, surface wettability and the length of the linking group which attaches the initiation site to the surface.

### SUMMARY OF THE INVENTION

The present invention provides a variety of methods for reducing non-specific binding of a target molecule or plurality of target molecules to an array of oligonucleotides.

As a first aspect, the present invention provides methods for reducing non-specific binding of a target molecule or detection/signal-generating molecule to a plurality of oligonucleotides on a surface of a solid support, wherein the surface has a plurality of designated regions and a plurality of protected regions. Each of the plurality of protected regions has a protecting group thereon. The method comprises: a) producing the plurality of oligonucleotides at each of the designated regions, each of the plurality of oligonucleotides having a terminal protecting group; and b) removing the protecting groups on each of the plurality of protected regions. Non-specific binding of a target molecule to the oligonucleotide array is reduced as a result of the removal of the protecting groups. In another embodiment, the methods of the present invention involve the removal of the protecting groups from both the plurality of oligonucleotides and the protected regions. The protecting groups may be photolabile protecting groups, or chemically-removable protecting groups, or combinations thereof.

As a second aspect, the present invention provides another method for reducing the non-specific binding of a target molecule or detection/signal-generating molecule to an oligonucleotide array. The method comprises a) producing the plurality of oligonucleotides at each of the designated regions, each of the plurality of oligonucleotides having a terminal protecting group; and b) replacing with a negatively charged phosphate residue, at least one of: i) the protecting groups on each of the plurality of oligonucleotides produced in step a), and ii) the protecting groups on each of the plurality of protected regions. Non-specific binding of the target molecule to the oligonucleotide array is reduced by the replacement of the protecting groups with a negatively charged phosphate residue. The present invention further comprises replacing the protecting groups on both the oligonucleotides and the protected regions. The negatively charged phosphate residue which replaces the protecting groups can be provided by reaction with a compound that covalently bonds a negatively charged phosphate residue or by attaching a polyanion chain of negatively charged phosphate residues.

As a third aspect, the present invention provides yet another method for reducing the non-specific binding of a target molecule to an oligonucleotide array. The method comprises: a) attaching a polyanion chain having a protecting group to each of the designated regions on the surface of the solid support, and b) forming the plurality of oligonucleotides having a terminal protecting group on the polyanion chains at each of the designated regions. Non-specific binding of a target molecule to the oligonucleotide array is reduced by the presence of the polyanion chains on the designated regions of the surface of the solid support. The polyanion chain may be attached to the designated regions by forming the polyanion chain on the designated regions or attaching a pre-formed polyanion chain to the designated regions. In one embodiment, a polyanion chain having a protecting group is also attached to the protected regions of the solid support.

In a fourth embodiment, the present invention provides a method for reducing non-specific binding of a target molecule to an oligonucleotide array. The method comprises a) attaching a polyanion chain having a protecting group to each of the protected regions and b) forming the plurality of oligonucleotides having a terminal protecting group at each of the designated regions. Non-specific binding of a target molecule to the oligonucleotide array is reduced by the presence of the polyanion chains on the protected regions of the surface of the solid support. The polyanion chain may be attached to the protected regions by forming the polyanion chain on the protected regions or attaching a pre-formed polyanion chain to the protected regions.

In a fifth embodiment, the present invention provides a method for reducing non-specific binding of a target molecule to an oligonucleotide array. The method includes a) attaching a polyanion chain having a protecting group to each of the designated regions, b) forming the plurality of oligonucleotides having a terminal protecting group, on the polyanion chains at each of the designated regions, and c) removing at least one of: i) the protecting groups on each of the plurality of oligonucleotides produced in step b), and ii) the protecting groups on each the plurality of protected regions. Non-specific binding of a target molecule to the oligonucleotide array is reduced by the combination of the polyanion chains on the designated regions and the removal of the protecting groups from either or both of the oligonucleotides or the protected regions of the surface of the solid support. In another embodiment, the polyanion chain is attached to the protected regions and non-specific binding of the target molecule to the oligonucleotide array is reduced by the combination of the polyanion chains on the protected regions and the removal of the protecting groups from either or both of the oligonucleotides or the polyanion chains attached to the protected regions of the surface of the solid support. In another embodiment, polyanion chains are attached to both the designated regions and the protecting regions and non-specific binding of the target molecule to the oligonucleotide array is reduced by the combination of the polyanion chains and the removal of the protecting groups from either or both of the oligonucleotides or the polyanion chains attached to the protected regions of the surface of the solid support.

In another embodiment, the present invention provides a method for reducing non-specific binding of a target molecule to an oligonucleotide array. The method includes a) attaching a polyanion chain having a protecting group to each of the designated regions; b) forming the plurality of oligonucleotides having a terminal protecting group on the polyanion chains at each of the designated regions, and c) replacing with a negatively charged phosphate residue, at least one, or both of: i) the protecting groups on each of the plurality of oligonucleotides produced in step b), and ii) the protecting groups on each the plurality of protected regions. Non-specific binding of a target molecule to the oligonucleotide array is reduced by the combination of the polyanion chains on the designated regions and the replacement of the protecting groups on either or both of the oligonucleotides or the protected regions of the surface of the solid support. In another embodiment, the polyanion chain is attached to the protected regions and non-specific binding of the target molecule to the oligonucleotide array is reduced by the combination of the polyanion chains on the protected regions and the replacement of the protecting groups on either or both of the oligonucleotides or the polyanion chains attached to the protected regions of the surface of the solid support. In another embodiment, polyanion chains are attached to both the designated regions and the protecting regions and non-specific binding of the target molecule to the oligonucleotide array is reduced by the combination of the polyanion chains and the replacement of the protecting groups on either or both of the oligonucleotides or the polyanion chains attached to the protected regions of the surface of the solid support.

In another embodiment, the present invention provides a solid support for solid-phase synthesis. The solid support comprises a surface having a plurality of designated regions and a plurality of protected regions and a polyanion chain having a protecting group attached to at least one of: i) each of the designated regions, and ii) each of the protected regions.

In yet another embodiment, the present invention provides a method for screening a target molecule for hybridization to a plurality of oligonucleotides. The method includes a) providing a solid support comprising a surface having a plurality of designated regions and a plurality of protected regions and a polyanion chain having a protecting group attached to at least one of: i) each of the designated regions, and ii) each of the protected regions, b) contacting the target molecule to the plurality of oligonucleotides; and c) detecting hybridization of the target molecule to the plurality of oligonucleotides. The oligonucleotides are attached to the polyanion chain when the polyanion chain is attached to each of the designated regions. Non-specific binding of a target molecule to the oligonucleotide array is reduced by the presence of the polyanion chains at one or both of the designated regions and the protected regions.

The foregoing methods may be combined in a variety of ways to reduce non-specific binding of a target molecule to an oligonucleotide array as well. Any suitable combination of the foregoing methods may be employed according to the present invention.

These and other aspects of the present invention are described further in the description of the preferred embodiment and the examples of the invention which follow.

### BRIEF DESCRIPTION OF THE DRAWINGS

FIG. 1 is an image obtained by the process of the present invention.

FIG. 2 is a control array wherein the substrate is not photolysed prior to hybridization.

FIG. 3 is an array wherein the substrate is modified according to the methods of the present invention prior to hybridization, in order to reduce specular non-specific binding.

### DESCRIPTION OF THE PREFERRED EMBODIMENT

Generally, the present invention relates to methods for reducing non-specific binding of a target molecule to an oligonucleotide array. Such methods include surface modifications to the substrate surface on which the oligonucleotides are provided as well as modifications to the oligonucleotides and combinations of these techniques.

As used herein, “EDA” means ethylenediamine; “AcOH” means acetic acid; “ALLOC” means allyloxycarbonyl; “BOP” means benzotriazol-1-yloxy-tris(dimethylamino)phosphonium hexafluorophosphate; “CAP” means E-aminocaproic acid; “DIEA” means diisopropylethylamine; “DIGLY” means glycylglycine; “DMF” means dimethylformamide; “DMT” means dimethoxytrityl; “DTT” means dithiothreitol; “EtOAc” means ethyl acetate; “FMOC” means fluorenylmethoxycarbonyl; “MeNPOC” means α-methylnitro-piperonyloxycarbonyl; “MP” means melting point; “NVOC” means nitroveratryloxycarbonyl; “OBt” means hydroxybenzotriazole radical; “PBS” means phosphate buffered saline; “TFA” means trifluoroacetic acid; “15-ATOM-PEG” means H₂N—(CH₂CH₂O)₂—CH₂CH₂NHCO—(CH₂)₃—CO₂H; and “TRIGLY” means glycylglycylglycine.

The following terms are intended to have the following general meanings as they are used herein:

Chemical terms: As used herein, the term “alkyl” refers to a saturated hydrocarbon radical which may be straight-chain or branched-chain (for example, ethyl, isopropyl, t-amyl, or 2,5-dimethylhexyl). When “alkyl” or “alkylene” is used to refer to a linking group or a spacer, it is taken to be a group having two available valences for covalent attachment, for example, —CH₂CH₂—, —CH₂CH₂CH₂—, —CH₂CH₂CH(CH₃)CH₂— and —CH₂(CH₂CH₂)₂CH₂—. Preferred alkyl groups as substituents are those containing 1 to 10 carbon atoms, with those containing 1 to 6 carbon atoms being particularly preferred. Preferred alkyl or alkylene groups as linking groups are those containing 1 to 20 carbon atoms, with those containing 3 to 6 carbon atoms being particularly preferred. The term “polyethylene glycol” is used to refer to those molecules which have repeating units of ethylene glycol, for example, hexaethylene glycol (HO—(CH₂CH₂O)₅—CH₂CH₂OH). When the term “polyethylene glycol” is used to refer to linking groups and spacer groups, it would be understood by one of skill in the art that other polyethers or polyols could be used as well (i.e, polypropylene glycol or mixtures of ethylene and propylene glycols).

The term “protecting group” as used herein refers to photolabile protecting groups and chemically-removable protecting groups.

The term “photolabile protecting group” as used herein refers to any of the groups which are designed to block one reactive site in a molecule while a chemical reaction is carried out at another reactive site, and which are removable by exposure to radiation such as light radiation. Specific examples of photolabile protecting groups within the meaning of that term as employed herein include but are not limited to groups such as dimethoxybenzoin, 2-nitroveratryloxycarbonyl (NVOC), α-methyl-2-nitroveratryloxycarbonyl (MeNVOC), 2-nitropiperonyloxycarbonyl (NPOC), a-methyl-2-nitropiperonyloxycarbonyl (MeNPOC), 2,6-dinitrobenzyloxycarbonyl (DNBOC), α-methyl-2,6-dinitrobenzyloxycarbonyl (MeDNBOC), 2-(2-nitrophenyl)ethyloxycarbonyl (NPEOC), 2-methyl-2-(2-nitrophenyl)ethyloxycarbonyl (MeNPEOC), 1-pyrenylmethyloxycarbonyl (PYMOC), 9-anthracenylmethyloxycarbonyl (ANMOC), 3′-methoxybenzoinyloxycarbonyl (MBOC), 3′,5′-dimethoxybenzoinyloxycarbonyl (DMBOC), 7-nitroindolinyloxycarbonyl (NIOC), 5-bromo-7-nitroindolinyloxycarbonyl (BNIOC), 5,7-dinitroindolinyloxycarbonyl (DNIOC), 2-anthraquinonylmethyloxycarbonyl (AQMOC), α,α-dimethyl-3,5-dimethoxybenzyloxycarbonyl, and non-carbonate benzylic forms of any of the foregoing e.g., NV, MeNV, and the like.

The term “chemically-removable protecting group” as used herein, refers to any of the groups which are designed to block one reactive site in a molecule while a chemical reaction is carried out at another reactive site, and which are removable by exposure to a chemical agent, that is by means other than exposure to radiation. For example, one type of chemically-removable protecting group is removably by exposure to a base (i.e., “base-removable protecting groups”). Examples of specific base-removable protecting groups include but are not limited to fluorenylmethyloxycarbonyl (FMOC), 2-cyanoethyl (CE), N-trifluoroacetylaminoethyl (TF), 2-(4-nitrophenyl)ethyl (NPE), and 2-(4-nitrophenyl)ethyloxycarbonyl (NPEOC). Another type of chemically removable protecting groups are removable by exposure to a nucleophile (i.e., “nucleophile-removable protecting groups”). Specific examples of nucleophile-removable protecting groups including but are not limited to levulinyl (Lev) and aryloxycarbonyl (AOC). Other chemically-removable protecting groups are removable by exposure to an acid (i.e., “acid-removable protecting groups”). Specific acid-removable protecting groups include but are not limited to triphenylmethyl (Tr or trityl), 4-methoxytriphenylmethyl (MMT or monomethoxytrityl), 4,4′-dimethoxytriphenylmethyl (DMT or dimethoxytrityl), tert-butoxycarbonyl (tBOC), α,α-dimethyl-3,5-dimethyoxybenyloxycarbonyl (DDz), 2-(trimethylsilyl)ethyl (TMSE), and 2-(trimethylsilyl)ethyloxycarbonyl (TMSEOC). Another type of chemically-removable protecting group is removable by exposure to a reductant (i.e., “reductant-removable protecting group”). Specific examples of reductant-removable protecting groups include but are not limited to 2-anthraquinonylmethyloxycarbonyl (AQMOC) and 2,2,2-trichloroethyloxycarbonyl (TROC). Additional examples of chemically-removable protecting groups include allyl (All) and allyloxycarbonyl (AllOC) protecting groups. Other examples of chemically-removable protecting groups will be known to those of skill in the art.

Monomer: A monomer is a member of the set of small molecules which are or can be joined together to form a polymer or a compound composed of two or more monomeric units. The present invention is described herein in terms of compositions and methods which are useful in solid phase synthesis. In a number of applications, solid phase methods are used for the preparation of biological polymers such as oligonucleotides. For these biological polymers, the set of monomers includes but is not restricted to, for example, the set of nucleotides and the set of pentoses and hexoses. The particular ordering of monomers within a biological polymer is referred to herein as the “sequence” of the polymer. As used herein, monomers refers to any member of a basis set for synthesis of a polymer. Different basis sets of monomers may be used at successive steps in the synthesis of a polymer. Furthermore, each of the sets may include protected members which are modified after synthesis. The invention is described herein primarily with regard to the preparation of molecules containing sequences of monomers such as nucleotides, but could readily be applied in the preparation of other polymers. Such polymers include, for example, both linear and cyclic polymers of nucleic acids, polysaccharides, phospholipids, and peptides having either α-, β-, or ω-amino acids, heteropolymers in which a known drug is covalently bound to any of the above, polynucleotides, polyurethanes, polyesters, polycarbonates, polyureas, polyamides, polyethyleneimines, polyarylene sulfides, polysiloxanes, polyimides, polyacetates, or other polymers which will be apparent upon review of this disclosure. Such polymers are “diverse” when polymers having different monomer sequences are formed at different designated regions of a substrate. Methods of cyclization and polymer reversal of polymers are disclosed in copending application U.S. Pat. No. 5,550,215 entitled “Polymer Reversal on Solid Surfaces,” incorporated herein by reference for all purposes.

Solid Support: As used herein, the term “solid support” or “substrate” refers to a material having a rigid or semi-rigid surface. In many embodiments, at least one surface of the support will be substantially flat, although in some embodiments it may be desirable to physically separate synthesis regions for different polymers with, for example, wells, raised regions, etched trenches, or the like. In some embodiments, the support itself contains wells, trenches, flow through regions, etc. which form all or part of the synthesis regions. According to other embodiments, small beads may be provided on the surface, and compounds synthesized thereon may be released upon completion of the synthesis.

Channel Block: A material having a plurality of grooves or recessed regions on a surface thereof. The grooves or recessed regions may take on a variety of geometric configurations, including but not limited to stripes, circles, serpentine paths, or the like. Channel blocks may be prepared in a variety of manners, including etching silicon blocks, molding or pressing polymers, etc.

Designated Region: A designated region is a localized area on the surface of a solid support which is, was, or is intended to be used for formation of a selected oligonucleotide polymer and is otherwise referred to herein in the alternative as “preselected” region, “predefined” region, “reaction” region, or “selected” region. The designated region may have any convenient shape, e.g., circular, rectangular, elliptical, wedge-shaped, etc. In some embodiments, a designated region and, therefore, the area upon which each distinct polymer sequence is synthesized is smaller than about 1 cm², more preferably less than 1 mm², and still more preferably less than 0.5 mm². In most preferred embodiments the designated regions have an area less than about 10,000 μm² or, more preferably, less than 100 μm². Within these regions, the polymer synthesized therein is preferably synthesized in a substantially pure form. Additionally, multiple copies of the polymer will typically be synthesized within any designated region. The number of copies can be in the thousands to the millions.

Protected Region: A protected region is a localized area on the surface of a solid support which is not used for formation of an oligonucleotide polymer. Thus, the protected regions are defined as all regions on the surface of the solid support where an oligonucleotide is not intended to be synthesized and which has attached thereto a protecting group. The protected region may have any convenient shape, e.g., circular, rectangular, elliptical, wedge-shaped, etc. In some embodiments, a protected region is smaller than about 1 cm², more preferably less than 1 m², and still more preferably less than 0.5 mm² In most preferred embodiments the protected regions have an area less than about 10,000 μm² or, more preferably, less than 100 μm².

Target Molecule: The term “target molecule” as used herein, refers to a molecule or plurality of molecules which hybridize to a plurality of oligonucleotides on an array and are thereby advantageously detected and/or identified. Typically, the target molecule(s) is contained in a mixture containing a variety of molecules, such as a biological sample. The term “biological sample”, as used herein, refers to a sample obtained from an organism or from components (e.g., cells) of an organism. The sample may be of any biological tissue or fluid. Frequently the sample will be a “clinical sample” which is a sample derived from a patient. Such samples include, but are not limited to, sputum, blood, blood cells (e.g., white cells), tissue or fine needle biopsy samples, urine, peritoneal fluid, and pleural fluid, or cells therefrom. Biological samples may also include sections of tissues such as frozen sections taken for histological purposes. The invention finds particular utility in assaying for target molecules in biological samples.

I. General Method of Assaying a Sample Containing Target Molecule(s)

The advent of methods for the synthesis of diverse chemical compounds on solid supports has resulted in the genesis of a multitude of research and diagnostic applications for such chemical libraries. A number of these research and diagnostic applications involve contacting a sample with a solid support, or chip, having multiple attached biological polymers such as peptides and oligonucleotides, or other small ligand molecules synthesized from building blocks in a stepwise fashion, in order to identify any species which specifically binds to one or more of the attached polymers or small ligand molecules.

For example, patent application Ser. No. 08/082,937, filed Jun. 25, 1993, describes methods for making arrays of oligonucleotide probes that can be used to provide the complete sequence of a target nucleic acid and to detect the presence of a nucleic acid containing a specific oligonucleotide sequence. U.S. Pat. No. 5,556,752 entitled “Surface Bound Unimolecular Double Stranded DNA,” describes methods of making arrays of unimolecular, double-stranded oligonucleotides which can be used in diagnostic applications involving protein/DNA binding interactions such as those associated with the p53 protein and the genes contributing to a number of cancer conditions. Arrays of double-stranded oligonucleotides can also be used to screen for new drugs having particular binding affinities.

The methods and compositions of the present invention are useful for reducing non-specific binding of one or more target molecules to an oligonucleotide array. These methods employ oligonucleotide arrays which comprise probes exhibiting complementarity to one or more selected target molecules whose sequence is known. Typically, these arrays are immobilized in a high density array (“DNA on chip”) on a solid support as described in U.S. Pat. No. 5,143,854 and PCT Publication Nos. WO 90/15070, WO 92/10092 and WO 95/11995, each of which is incorporated herein by reference. Generally speaking, a sample containing one or more target molecules can be assayed or screened against a plurality of oligonucleotides on an array to detect and/or identify the target molecule in the sample. The method involves providing the solid support having the plurality of oligonucleotide probes thereon, contacting the plurality of target molecules to the oligonucleotide array, and detecting hybridization of the target molecules to the plurality of oligonucleotides. The hybridization and assay techniques are described in copending U.S. patent application Ser. No. 08/797,812, filed Feb 2, 1997, incorporated herein by reference in its entirety. Expression analysis techniques are described in PCT publication WO-97/10365, incorporated by reference in its entirety.

Various strategies are available to order and display the oligonucleotide arrays on the chip and thereby maximize the hybridization pattern and sequence information derivable regarding the target nucleic acid. Exemplary display and ordering strategies, including the basic tiling strategy, are described in PCT Publication No. WO 94/12305, incorporated herein by reference, and U.S. patent application Ser. No. 08/797,812, filed Feb. 2, 1997 [18547-018550]. Techniques for exposing one or more target molecules to the oligonucleotide array to determine hybridization of the target molecule(s) to the oligonucleotide array are known in the art.

Non-specific binding to the oligonucleotide array occurs when molecules which are not target molecules appear to be hybridized to the array. The result of non-specific binding is decreased accuracy in the qualitative and/or quantitative measurement of the target molecule in the sample. Non-specific binding can result from a number of factors. Non-specific binding appears to arise from binding of protein components within the sample, such as fluorescein- or phycoerythrin-streptavidin to the oligonucleotide array. In other cases, bright “speckles” occur which appear to be caused by the adhesion of precipitated particles of insoluble magnesium, cobalt, or other salts present in the buffers used for RNA fragmentation or TdT labeling. Both kinds of non-specific binding are most severe in regions of the array or chip which possess unreacted protecting groups (e.g., MeNPOC-protecting groups) which have not been photolysed prior the final substrate deprotection in EDA. Areas of the chip on which oligonucleotide probes have been synthesized generally show the lowest intensity fluorescent non-specific binding. Unphotolysed surface linker sites are known to react with EDA to produce a mixture of free terminal hydroxyl groups as well as aminoethylcarbamoyl residues. The latter impart a net positive charge to the surface regions so treated, which contrasts with the probe-containing regions of the array which are negative. Although not wishing to be bound by any particular theory of the invention, it is believe that the net positive charges at these regions contribute to non-specific binding.

III. The Solid Support

The solid support may be biological, nonbiological, organic, inorganic, or a combination of any of these, existing as particles, strands, precipitates, gels, sheets, tubing, spheres, containers, capillaries, pads, slices, films, plates, slides, etc. The solid support is preferably flat but may take on alternative surface configurations. For example, the solid support may contain raised or depressed regions on which synthesis takes place. In some embodiments, the solid support will be chosen to provide appropriate light-absorbing characteristics. For example, the support may be a polymerized Langmuir Blodgett film, functionalized glass, fused silica, Si, Ge, GaAs, GaP, SiN₄, metal oxide films such as alumina or silicon dioxide, modified silicon, or any one of a variety of gels or polymers such as (poly)tetrafluoroethylene, (poly)vinylidenedifluoride, polystyrene, polycarbonate, or combinations thereof. Other suitable solid support materials will be readily apparent to those of skill in the art. Preferably, the surface of the solid support will contain reactive groups, which could be carboxyl, amino, hydroxyl, thiol, or the like. More preferably, the surface will be optically transparent and will have surface Si—OH functionalities, such as are found on silica surfaces.

The surface of the solid support is divided into essentially two regions, a plurality of designated regions and a plurality of protected regions. An oligonucleotide is, was, or will be attached or synthesized at each of the plurality of designated regions. The protected regions of the surface are distinct from the designated regions on the surface of the solid support. Each of the plurality of the protected regions on the surface are regions where the oligonucleotides are neither attached nor synthesized, and which have attached thereto, a removable protecting group. The protecting groups at each of the protected regions are preferably standard, photolabile or chemically-removable protecting groups including those groups which are commercially available and which are known to be removable by exposure to radiation or chemical conditions. Examples of such protecting groups include FMOC, DMT, NVOC, MeNPOC, BOC, ALLOC, t-butyl esters and t-butyl ethers. Currently, the preferred protecting groups for the protected regions are photolabile protecting groups such as NVOC, MeNVOC, or MeNPOC groups.

According to one embodiment of the invention, the surface of the solid support may be derivatized prior to the synthesis of the oligonucleotides thereon by the attachment of a linker monomer at each of the designated regions on the surface of the solid support. Derivatization with a linker monomer is not essential to carrying out the methods of the present invention. In particular, in those embodiments wherein a polyanion chain is attached to the designated regions of the surface of the solid support, the substrate is preferably not derivatized with the linker monomer prior to synthesis of the oligonucleotides.

In the embodiment wherein the surface of the solid support is derivatized with the linker monomer, each of the oligonucleotides is synthesized on the linker monomers by application of the general techniques described above. The linker monomers are attached to the surface of the solid support by reacting the support with a derivatization reagent having a substrate attaching group on one end and a reactive site on a distal end (away from the surface) to provide a support surface having an even distribution of reactive sites. The derivatized surface is then contacted with the linker molecules. The linker molecules each have reactive groups which are capable of covalent attachment to the reactive sites on the derivatized surface. The linker molecules additionally have a functional group which is protected with a protecting group. The contact is carried out for a sufficient period of time to bind the linker molecules to the surface of the solid support.

The derivatization reagent can be attached to the solid support via carbon-carbon bonds using, for example, solid supports having (poly)trifluorochloroethylene surfaces, or more preferably, by siloxane bonds (using, for example, glass or silicon oxide as the solid support). Siloxane bonds with the surface of the support are formed in one embodiment via reactions of derivatization reagents bearing trichiorosilyl or trialkoxysilyl groups.

The particular derivatization reagent used can be selected based upon its hydrophilic/hydrophobic properties to improve presentation of an attached oligonucleotide to certain receptors, proteins or drugs. As noted above, the derivatization reagent, prior to attachment to the solid support, has a substrate attaching group at one end, and a reactive site at the other end. The reactive site will be a group which is appropriate for attachment to a linker monomer, nucleotide monomer, or negatively-charged phosphate monomer. For example, groups appropriate for attachment to a silica surface would include trichlorosilyl and trialkoxysilyl functional groups. Groups which are suitable for attachment to a linker monomer, nucleotide monomer or negatively-charged phosphate monomer include amine, hydroxyl, thiol, carboxylic acid, ester, amnide, epoxide, isocyanate and isothiocyanate. Additionally, for use in synthesis, the derivatization reagents used herein will typically have a protecting group attached to the reactive site on the distal or terminal end of the derivatization reagent (opposite the solid support). Preferred derivatization reagents include aminoalkyltrialkoxysilanes, aminoalkyltrichlorosilanes, hydroxyalkyltrialkoxy-silanes, hydroxyalkyltrichlorosilanes, carboxyalkyltrialkoxysilanes, polyethyleneglycols, triethoxysilane, epoxyalkyltrialkoxysilanes, and combinations thereof.

In the embodiment wherein a linker monomer is employed at each of the designated regions, the derivatized surface is contacted with the linker monomers. The linker monomers have one center which is reactive with the reactive sites on the derivatized surface of the solid support. Additionally, the linker monomers will have a functional group which is protected with a protecting group and which can later serve as a synthesis initiation site.

The linker monomers used in the present invention are preferably of sufficient length to permit any oligonucleotides synthesized thereon to interact freely with target molecules exposed to the oligonucleotides. The linker monomers should be 3-50 atoms long to provide sufficient exposure of ligands to their receptors. Typically, the linker monomers will be alkylene (—CH₂)ₙ—), aryl acetylene, ethylene glycol oligomers containing 2-14 monomer units, diamines, diols, diacids, amino acids, peptides, or combinations thereof. In some embodiments, the linker monomers can be a polynucleotide. The particular linking molecule used can be selected based upon its hydrophilic/hydrophobic properties to improve presentation of the polymer synthesized thereon to certain receptors, proteins or drugs. As noted above, the linker monomers, prior to attachment to the derivatized surface has an appropriate functional group at each end, one group appropriate for attachment to the reactive sites on a derivatized surface and the other group appropriate as a synthesis initiation site. For example, groups appropriate for attachment to the derivatized surface would include amino, hydroxy, thiol, carboxylic acid, ester, amide, isocyanate and isothiocyanate. Additionally, for subsequent use in synthesis of oligonucleotide arrays or libraries, the linker monomers used herein will typically have a protecting group attached to the functional group on the distal or terminal end of the linker monomer (opposite the solid support).

The linker monomer contributes to the net hydrophobic or hydrophilic nature of the surface. For example, when the linker monomers comprise a hydrocarbon chain, such as —(CH₂)ₙ—, the effect is to decrease wettability. Linker monomers including polyoxyethylene (—(CH₂CH₂O)ₙ—), or polyamide (—(CH₂CONH)ₙ—) chains tend to make the surface more hydrophilic thereby increasing wettability. In one preferred embodiment, the linker monomers comprise polyethyleneglycol molecules having protecting groups thereon. The protecting groups on the linker monomers are preferably standard, photolabile or chemically-removable protecting groups including those groups which are commercially available and which are known to be removable under typical chemical conditions. Examples of such protecting groups include FMOC, DMT, NVOC, MeNPOC, BOC, ALLOC, t-butyl esters and t-butyl ethers. Currently, the preferred protecting groups for the linker monomers photolabile groups such as NVOC, MeNVOC, MeNPOC, or DMT groups or chemically-removable groups.

IV. General Solid Phase Synthesis Techniques

The oligonucleotide probes may be synthesized at the designated regions on the solid support to produce the oligonucleotide array using any of various conventional techniques, including light-directed methods, flow channel and spotting methods, pin-based methods and bead-based methods. These methods of solid phase synthesis are described in, e.g. detail in U.S. Pat. Nos. 5,624,711, and 5,677,195 the disclosures of which are incorporated herein by reference in their entirety. Some of these techniques are described briefly hereinbelow.

“Light-directed” methods (which are one technique in a family of methods known as VLSIPST methods) are described in U.S. Pat. No. 5,143,854, previously incorporated by reference. The light directed methods discussed in the '854 patent involve activating the designated regions of a substrate or solid support and then contacting the surface with a preselected monomer solution. The designated regions can be activated with a light source, typically shown through a mask (much in the manner of photolithography techniques used in integrated circuit fabrication). Other regions of the surface remain inactive because they are blocked by the mask from illumination and thus remain chemically protected. In this manner, a light pattern defines which regions of the substrate react with a given monomer. By repeatedly activating different sets of predefined, designated regions and contacting different monomer solutions with the substrate, a diverse array of polymers is produced on the substrate. Of course, other steps such as washing unreacted monomer solution from the substrate can be used as necessary.

Additional methods applicable to library synthesis on a single substrate are described in U.S. Pat. Nos. 5,384,261 and 5,677,195, incorporated herein by reference for all purposes. In the methods disclosed in these applications, reagents are delivered to the support by either (1) flowing within a channel defined on predefined regions or (2) “spotting” on predefined regions. However, other approaches, as well as combinations of spotting and flowing, or the use of photoresist may be employed. In each instance, certain activated regions of the surface are mechanically separated from other regions when the monomer solutions are delivered to the various reaction sites.

A typical “flow channel” method applied to the compounds and libraries of the present invention can generally be described as follows. Diverse polymer sequences are synthesized at designated regions of a substrate or solid support by forming flow channels on a surface of the support through which appropriate reagents flow or in which appropriate reagents are placed. For example, assume a monomer “A” is to be bound to the substrate in a first group of selected regions. If necessary, all or part of the surface of the substrate in all or a part of the selected regions is activated for binding by, for example, flowing appropriate reagents through all or some of the channels, or by washing the entire substrate with appropriate reagents. After placement of a channel block on the surface of the support, a reagent having the monomer A flows through or is placed in all or some of the channel(s). The channels provide fluid contact to the first designated regions, thereby binding the monomer A on the surface directly or indirectly (via a spacer) in the first designated regions.

Thereafter, a monomer B is coupled to second designated regions, some of which may be included among the first designated regions. The second designated regions will be in fluid contact with a second flow channel(s) through translation, rotation, or replacement of the channel block on the surface of the support; through opening or closing a selected valve; or through deposition of a layer of chemical or photoresist. If necessary, a step is performed for activating at least the second regions. Thereafter, the monomer B is flowed through or placed in the second flow channel(s), binding monomer B at the second designated locations. In this particular example, the resulting sequences bound to the support at this stage of processing will be, for example, A, B, and AB. The process is repeated to form a vast array of sequences of desired length at known locations on the surface of the support. After the substrate is activated, monomer A can be flowed through some of the channels, monomer B can be flowed through other channels, a monomer C can be flowed through still other channels, etc. In this manner, many or all of the reaction regions are reacted with a monomer before the channel block must be moved or the substrate must be washed and/or reactivated. By making use of many or all of the available reaction regions simultaneously, the number of washing and activation steps can be minimized.

One of skill in the art will recognize that there are alternative methods of forming channels or otherwise protecting a portion of the surface of the support. For example, according to some embodiments, a protective coating such as a hydrophilic or hydrophobic coating (depending upon the nature of the solvent) is utilized over portions of the substrate to be protected, sometimes in combination with materials that facilitate wetting by the reactant solution in other regions. In this manner, the flowing solutions are further prevented from passing outside of their designated flow paths.

The “spotting” methods of preparing compounds and libraries of the present invention can be implemented in much the same manner as the flow channel methods. For example, a monomer A can be delivered to and coupled with a first group of reaction regions which have been appropriately activated. Thereafter, a monomer B can be delivered to and reacted with a second group of activated reaction regions. Unlike the flow channel embodiments described above, reactants are delivered by directly depositing (rather than flowing) relatively small quantities of them in designated regions. In some steps, of course, the entire surface of the support can be sprayed or otherwise coated with a solution. In preferred embodiments, a dispenser moves from region to region, depositing only as much monomer as necessary at each stop. Typical dispensers include a micropipette to deliver the monomer solution to the surface and a robotic system to control the position of the micropipette with respect to the surface, or an inkjet printer. In other embodiments, the dispenser includes a series of tubes, a manifold, an array of pipettes, or the like so that various reagents can be delivered to the reaction regions simultaneously.

Another method which is useful for the preparation of compounds and libraries of the present invention involves “pin based synthesis.” This method is described in detail in U.S. Pat. No. 5,288,514, previously incorporated herein by reference. The method utilizes a substrate having a plurality of pins or other extensions. The pins are each inserted simultaneously into individual reagent containers in a tray. In a common embodiment, an array of 96 pins/containers is utilized.

Each tray is filled with a particular reagent for coupling in a particular chemical reaction on an individual pin. Accordingly, the trays will often contain different reagents. Since the chemistry disclosed herein has been established such that a relatively similar set of reaction conditions may be utilized to perform each of the reactions, it becomes possible to conduct multiple chemical coupling steps simultaneously. In the first step of the process the invention provides for the use of substrate(s) on which the chemical coupling steps are conducted. The substrate is optionally provided with a spacer having active sites. In the particular case of oligonucleotides, for example, the spacer may be selected from a wide variety of molecules which can be used in organic environments associated with synthesis as well as aqueous environments associated with binding studies. Examples of suitable spacers are polyethyleneglycols, dicarboxylic acids, polyamines and alkylenes, substituted with, for example, methoxy and ethoxy groups. Additionally, the spacers will have an active site on the distal end. The active sites are optionally protected initially by protecting groups. Among a wide variety of chemically-removable protecting groups which are useful are FMOC, BOC, t-butyl esters, t-butyl ethers, and the like. Various exemplary protecting groups are described in, for example, Atherton et al., Solid Phase Peptide Synthesis, IRL Press (1989), incorporated herein by reference. In some embodiments, the spacer may provide for a cleavable function by way of, for example, exposure to acid or base.

Yet another method which is useful for synthesis of polymers and small ligand molecules on a solid support is “bead based synthesis.” A general approach for bead based synthesis is described in U.S. Pat. No. 5,541,061, the disclosure of which is incorporated herein by reference.

For the synthesis of molecules such as oligonucleotides on beads, a large plurality of beads are suspended in a suitable carrier (such as water) in a container. The beads are provided with optional spacer molecules having an active site. The active site is protected by an optional protecting group.

In a first step of the synthesis, the beads are divided for coupling into a plurality of containers. For the purposes of this brief description, the number of containers will be limited to three, and the monomers denoted as A, B, C, D, E, and F. The protecting groups are then removed and a first portion of the molecule to be synthesized is added to each of the three containers (i.e., A is added to container 1, B is added to container 2 and C is added to container 3).

Thereafter, the various beads are appropriately washed of excess reagents, and remixed in one container. Again, it will be recognized that by virtue of the large number of beads utilized at the outset, there will similarly be a large number of beads randomly dispersed in the container, each having a particular first portion of the monomer to be synthesized on a surface thereof.

Thereafter, the various beads are again divided for coupling in another group of three containers. The beads in the first container are deprotected and exposed to a second monomer (D), while the beads in the second and third containers are coupled to molecule portions E and F respectively. Accordingly, molecules AD, BD, and CD will be present in the first container, while AE, BE, and CE will be present in the second container, and molecules AF, BF, and CF will be present in the third container. Each bead, however, will have only a single type of molecule on its surface. Thus, all of the possible molecules formed from the first portions A, B, C, and the second portions D, E, and F have been formed.

The beads are then recombined into one container and additional steps are conducted to complete the synthesis of the polymer molecules. In a preferred embodiment, the beads are tagged with an identifying tag which is unique to the particular double-stranded oligonucleotide or probe which is present on each bead. A complete description of identifier tags for use in synthetic libraries is provided in European Patent Publication No. 0604552 (filed 16 September 1992).

The general light directed method for polymer synthesis on the solid support is currently one preferred method for synthesizing the oligonucleotides on the solid support. A variation on this general technique is also described in U.S. Pat. No. 5,624,711, already incorporated herein by reference in its entirety. Briefly, this technique for oligomer synthesis involves constructing a solid support having designated regions having attached thereto, photolabile protecting groups such that a protecting group is located at each of the designated regions. Using photolithographic techniques described in the above-noted General Methods section, the photolabile protecting groups can be removed in one designated area and a nucleotide monomer bearing a chemically-removable protecting group is attached using of phosphoramidite chemistry for monomer coupling. While this particular embodiment of the invention illustrates the use of phosphoramidite chemistry for monomer coupling, monomers can also be added to the growing oligomer using H-phosphonate methods or other coupling methods known to those of skill in the art.

Standard, chemically-removable protecting groups include those groups which are commercially available and which are known to be removable under typical chemical conditions such exposure to acid, base, oxidant, reductant or other chemical agent. Examples of such protecting groups include FMOC, DMT, BOC, t-butyl esters and t-butyl ethers. Following the attachment of such a protected nucleotide monomer, the protecting group is removed under conditions described in, for example, Greene, et al., Protective Groups In Organic Chemistry, 2nd Ed., John Wiley & Sons, New York, N.Y., 1991, previously incorporated herein by reference. The reactive functionality which was previously protected with the chemically-removable protecting group is then re-protected with a photolabile protecting group, using, for example, a derivative of the formula:

R—O—C(O)—X

in which R is a photolabile moiety (e.g., o-nitrobenzyls, including 2-nitroveratryloxycarbonyl, α-methyl-2-nitroveratryloxycarbonyl, 2-nitropiperonyloxycarbonyl, α-methyl-2-nitropiperonyloxycarbonyl, 2,6-dinitrobenzyloxycarbonyl, α-methyl-2,6-dinitrobenzyloxycarbonyl, 2-(2-nitrophenyl)ethyloxycarbonyl, 2-methyl 2-(2-nitrophenyl)ethyloxycarbonyl, 1-pyrenylmethyloxycarbonyl, 9-anthracenylmethyloxycarbonyl, 3′-methoxybenzoinyloxycarbonyl, 3′,5′-dimethoxybenzoinyloxycarbonyl, 7-nitroindolinyloxycarbonyl, 5-bromo-7-nitroindolinyloxycarbonyl, 5,7-dinitroindoinyloxycarbonyl, 2-anthraquinonylmethyloxycarbonyl) and X is a suitable leaving group (e.g., Cl, F, pentafluorophenoxy, p-nitrophenoxy, N-succinimidyloxy, adamantanecarboxy, or tetrazolyl). Preferably the derivative is a suitably activated derivative of the MeNPOC, MeNVOC or NVOC groups. Examples of suitably activated derivatives include such reagents as mixed anhydride derivatives of MeNPOC (e.g., MeNPOC-pivaloate prepared from the reaction of MeNPOC chloride with triethylammonium pivaloate) or carbonates of MeNPOC (e.g., the carbonate produced by the reaction of MeNPOC chloride with pentafluorophenol).

The re-protection of surface functional groups with such reagents is typically carried out in an organic solvent containing a non-nucleophilic base (e.g. 2,6-lutidine, pyridine, triethylamine or diisopropylethylamine). In some embodiments, a nucleophilic catalyst (e.g., N-methylimidazole, hydroxybenzotriazole or 4-(N,N-dimethylamino)pyridine) is also included to provide further enhancement of the rate and efficiency of the re-protection step.

Following the addition of the photolabile protecting groups, the VLSIPS cycles can be continued using photolithographic deprotection, followed by coupling of an additional nucleotide monomer, protecting group replacement, etc., until the desired oligomer are completed. Preferably, the cycle is repeated from 1 to 120 times.

The photolabile protecting group which is illustrated (MeNPOC) can be replaced with another photolabile protecting group such as NVOC, or other conventionally known photolabile protecting groups. Once the chemically-removable protecting group has been removed, a photolabile protecting group can be added using a mixed anhydride of the protecting group. The present method provides certain advantages over conventional VLSIPS synthesis. For example, a number of monomers having chemically-removable protecting groups are commercially available.

In the embodiment wherein the linker monomer is attached to the designated regions, the foregoing methods for oligonucleotide synthesis are equally applicable. For example, the nucleotide monomers are reacted and thereby attached to the linker monomers by replacing chemically-removable protecting groups in each of the linker monomers with a photolabile protecting group and attaching the nucleotide monomers at the location of the photolabile protecting group by using the light-directed methods described above for the attachment of each nucleotide monomer to the previous nucleotide monomer. As noted above, each nucleotide monomer will have a chemically-removable protecting group which permits the attachment of another nucleotide monomer by replacement with a photolabile protecting group and reaction using light-directed methods. Any remaining functional groups on the nucleotide monomers are capped or protected.

Another variation on the general light directed techniques include the methods described in co-pending application Ser. No. 08/969,227, filed Nov. 13, 1997, the subject matter of which is incorporated by reference in its entirety for all purposes. Briefly, this method employs nucleotide monomers having chemically-removable protecting groups, such as those described above, for the production of the oligonucleotides on the surface. According to this method, radiation signals are detected by a catalyst system including, for example, a photoactive agent, which is a photo activated catalyst (PAC). The photoactive agent activates an autocatalytic agent (or “enhancer”), which increases the effective quantum yield of the photons in subsequent chemical reactions. Such subsequent reactions include the removal of chemically-removable protective groups in the synthesis of patterned arrays.

In certain embodiments, a photoacid generator (i.e., a photo activated acid catalyst (PAAC)) is irradiated. The resulting acid produced from the photoacid generator activates the autocatalytic agent to undergo an acid-catalyzed reaction to itself produce an acid that removes acid labile protecting groups from the attached molecule or oligomer.

According to one embodiment of this method, monomers having chemically-removable protecting groups are provided on the surface of a substrate. The monomers may be linker monomers, nucleotide monomers, or monomers having a negatively charged phosphate unit and a protecting group. An activation layer (or “catalyst system”) including a photoactive agent and an autocatalytic agent are also provided on the surface. A set of selected regions on the surface of the substrate is exposed to radiation using well-known lithographic methods discussed, for example, in Thompson, L. F., Willson, C. G, and Bowden, M. J., INTRODUCTION TO MICROLITHOGRAPHY, American Chemical Society, pp. 212-232 1994, incorporated herein by reference in its entirety for all purposes.

The photoactive agent activated by the region-selective irradiation acts to initiate a reaction of the autocatalytic agent. The autocatalytic agent produces at least one product that removes the protecting groups from the molecules or oligomers attached to the substrate in the first selected regions. Preferably, the autocatalytic agent is capable of removing protecting groups in a catalytic manner. The substrate is then washed or otherwise contacted with the next monomer to be added which then reacts with exposed functional groups on the previously added monomer.

Thereafter, a second set of selected regions is exposed to radiation. The radiation-initiated reactions remove the protecting groups on molecules in the second set of selected regions. The substrate is then contacted with the next monomer to be added, which has chemically-removable protecting groups for reaction with exposed functional groups. This process is repeated to selectively apply monomers until polymers (e.g., oligonucleotides) of a desired length and desired chemical sequence are obtained. Protecting groups are then optionally removed and the sequence is, thereafter, optionally capped. Side chain protecting groups, if present, are also optionally removed.

In other embodiments of this method, the chemically-removable protecting groups are removed by the exposure to a base, or an oxidant, or a reductant. In each embodiment, the photoactive agent and the autocatalytic agent are such that the appropriate chemical reagent for removal of the given protecting group is generated and the protecting groups are thereby removed. Generation of base, oxidant or reductant can be achieved using photoactive agents which are conventionally known in the art of photoresist technology and applying the guidelines provided above in the example of generating an acid catalyst.

Yet another improvement on the general light directed methods of producing oligonucleotide polymers on a substrate is described in U.S. Pat. No. 5,658,734 to Phillip Brock et al., the disclosure of which is hereby incorporated by reference in its entirety for all purposes. This method also employs nucleotide monomers having chemically-removable protecting groups for the production of the oligonucleotides on the surface. Briefly, the method includes coating a layer of developable polymer onto a layer of foundational molecules (e.g., a first nucleotide monomer having chemically-removable protecting group, or a linker monomer having a chemically-removable protecting group, or a polyanion chain having a chemically-removable protecting group) which is attached to the substrate. The foundational molecules may be attached to the substrate through a linker monomer or a polyanion chain. Suitable polymers for use in this method include soluble polyimides and poly(vinylalcohol) (PVA). The developable polymer is dissolved in a suitable coating solvent such as anisole, water (for PVA), N-methyl pyrrolidone or N,N-dimethylacetamide.

The developable polymer can be coated on the layer of foundational molecules using art-known techniques such as spin or spray coating, or doctor blading. Preferably, the polymer layer is then heated to remove the casting solvent.

Thereafter, a polymeric, radiation sensitive resist is coated onto the developable polymer layer in a manner similar to the application of the developable polymer layer. Preferred resists are crosslinking negative tone resists which are resistant to the organic solvents utilized to transfer the lithographic pattern from the resist through the developable polymer. Suitable resists include crosslinking epoxy resists, cyclized rubber resists such as KTFR, polyvinylcinnamate resists, such as KPR, and those resists described in W. DeForest, PHOTORESIST MATERIALS AND PROCESSES, McGraw-Hill, New York 1975 and W. Moreau, SEMICONDUCTOR LITHOGRAPHY, Plenum Press, New York 1988.

After application, selection regions of the resist layer are exposed to radiation, and the latent image in the resists is developed with a suitable developer solvent, many of which are known in the art of photoresist lithography. The image of radiation exposure is developed through the resist layer and the developable polymer layer to the underlying layer of foundational molecules through the use of suitable solvents. The thus, uncovered portions of the underlying layer of foundational molecules are then treated to remove the chemically-removable protecting group from the exposed molecules and thereby activate the exposed foundational molecules. Preferably, the exposed foundational molecules are treated with a solution to cleave the protecting group from each of the molecules. Examples of suitable cleaving solutions include strong acids, and solutions of ammonia, amines, or other strong bases as well as oxidants and reductants.

After removal of the chemically-removable protecting groups on the exposed foundational molecules, the remaining portions of the resist layer and developable polymer layer are removed. Subsequently, the next monomer is applied, which bonds to the exposed, unprotected, foundational molecules. The oligomers produced by the addition of the second monomer to the foundational molecules then become the foundation for the next iteration of the process. These steps are repeated until the desired number of monomeric units are joined to provide the polymers.

Each of the foregoing methods exemplify the general techniques which can be employed by the skilled artisan for the production of the oligonucleotides on the surface of the solid support. As will be apparent to those skilled in the art, it is also possible to utilize different types of protecting groups on the monomers. For example, linking monomers may utilize photolabile protecting groups while nucleotide monomers utilize chemically-removable protecting groups. Protecting groups on the protected regions may be chemically removable protecting groups while protecting groups on linker and nucleotide monomers are photolabile protecting groups. There is no requirement in any of the foregoing processes that all protecting groups must be the same. Any of the foregoing techniques may be employed for removing different types of protecting groups by the combination of different activators as is necessary. Such analogous combinations of the foregoing methods is within the skill of those in the art.

VI. Surface Modification

The methods of the present invention for reducing non-specific binding to an oligonucleotide array in one embodiment involves surface modification of the solid support. Several methods of surface modification may be employed in order to reduce non-specific binding of target molecules to the oligonucleotide array.

Removal of Protecting Groups from the Protected Regions

In one embodiment of the present invention, the non-specific binding to the oligonucleotide array is reduced by removing the protecting groups at the protected regions on the surface of the solid support. The protecting groups on the protected regions of the surface may be removed by exposing each of the protected regions to a suitable activator to remove the protecting group from each of the plurality of protected regions. The activators listed above for use in oligonucleotide synthesis may be employed for the removal of the protecting groups from the protected regions.

Without wishing to be bound by any particular theory, it is believed that the removal of the protecting group from each of the plurality of protected regions reduces non-specific binding of target molecules to the oligonucleotide array because of a change in polarity on the surface of the solid support, which change is brought about by the removal of these protecting groups. More specifically, the removal of the protecting groups removes the positive charge carried by these protecting groups, which is believed to be a contributing factor in non-specific binding. The removal of this positive charge thereby removes one contributing factor to non-specific binding.

Replacing Protecting Groups on Protected Regions

In another embodiment of the present invention, the surface of the solid support is modified by replacing the protecting groups on the protected regions with a negatively charged phosphate residue. The protecting groups on the protected regions of the surface may advantageously be replaced with a negatively charged phosphate residue by exposing the plurality of protected regions to an activator to produce activated sites and reacting the activated sites with a compound that covalently bonds a negatively charged phosphate residue to each of the plurality of protected regions. The activator employed may be any of the suitable activators described above for use in oligonucleotide synthesis and removal of protecting groups. The compounds which are reacted with the activated sites are selected from the group consisting of Formula I:

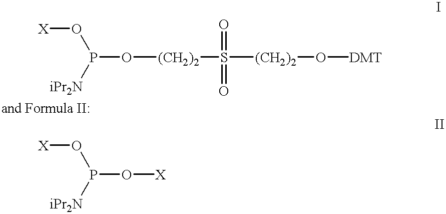

wherein DMT is a dimethoxy trityl protecting group, X is a base-removable protecting group (e.g., 2-cyanoethyl), and iPr₂N is diisopropyl amino protecting group.

Advantageously compounds of Formula I or II are commercially available. Examples of suitable compounds within the scope of Formulas I or II include but are not limited to compounds defined where X is 2-cyanoethyl. These compounds are preferred for use in the methods of replacing the protecting groups on the protected regions of the surface. The reaction of these compounds with the activated site can be carried out by contacting the activated sites on the surface of the solid support to such compounds in the solution phase for a period of time sufficient to effect the covalent attachment of the compounds of Formula I or II to the protected regions. Suitable reagents for use in this method include but are not limited to 0.45M tetrazole in anhydrous acetonitrile. Preferably, the compounds of Formula I or II are reacted with the activated sites for a period of from about 0.5 to about 15 minutes. The techniques employed are the same as or analogous to the techniques described above for oligonucleotide synthesis.

In one embodiment of the present invention, the surface of the substrate is modified to provide a monolayer of cytidine at one or both of the designated regions and the protected regions on the surface of the substrate.

In another embodiment, the protecting groups on the protected regions of the surface may be replaced by attachment of a polyanion chain. Methods for the attachment of a polyanion chain to either or both of the protected regions and the designated regions on the surface of the solid support are described below.

Attachment of Polyanion Chains

According to another method of the present invention, the surface of the solid support is derivatized with a polyanion chain. The polyanion chain may be attached in place of the linker monomers at the designated regions of the surface, where the oligonucleotides will be attached. In this embodiment, rather than providing a linker monomer at each of the designated regions, a polyanion chain would be provided instead and the oligonucleotides would be attached to or synthesized on each of the polyanion chains at the designated regions of the surface. In another embodiment, the polyanion chain is attached only at each of the protected regions on the surface of the solid support. In this embodiment, the designated regions of the surface would preferably have attached thereto the linker monomers as described above. In yet a third embodiment, the polyanion chain may be attached to both i) each of the designated regions and ii) each of the protected regions. The attachment of the polyanion chains to either or both of the designated regions and the protected regions on the surface of the solid support has the effect of reducing non-specific binding of a target molecule to the oligonucleotide probes during a hybridization assay. In one preferred embodiment of the methods of the present invention, at least one of i) the designated regions and the ii) protected regions have polyanion chains attached thereto such that the surface of the solid support is modified to include polyanion chains at one or both of the designated regions and the protected regions.

The polyanion chain may be attached to the desired region(s) on the surface by either synthesizing or forming the polyanion chain at the desired region(s) or attaching a pre-formed polyanion chain to the desired region(s). The surface of the solid support may be prepared for attachment of the polyanion chain by utilizing a derivatizing reagent in the manner discussed above for the attachment of a linker monomer. In the first embodiment, the polyanion chains are formed at the desired region(s) by attaching a monomer having a negatively charged phosphate unit and a protecting group to the desired region(s), that is to at least one of i) each of the designated regions on the surface or ii) each of the protected regions on the surface or both, so that the monomer is covalently bound to the desired region(s). Thereafter, the monomer is exposed to an activator to remove the protecting group, thereby producing activated sites which are capable of reacting with additional monomers to add monomeric units to the first monomer, thereby building the polymer chain. The steps of exposing the monomer to an activator to produced activated sites and reacting with additional monomer are repeated from 0-20 times to produce the polyanion chain at the desired regions.

The monomers which are employed to build the polyanion chain advantageously include any commercially available monomers having negatively charged phosphate units and a photolabile or chemically-removable protecting group, and which are capable of attachment to the surface of the particular solid support selected. For example, a suitable monomer for forming the polyanion chain at the desired region(s) is a monomer of Formula III:

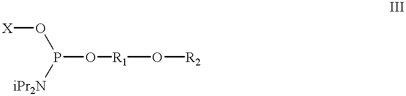

wherein R₁ is selected from the group consisting of a nucleoside moiety, a deoxyribose moiety, C₁₋₈ alkylene, and —(CH₂CH₂O)ₘ— wherein m is an integer from 1 to 8; R₂ is selected from the group consisting of a dirnethoxy trityl protecting group and a MeNPOC protecting group; X is a base-removable protecting group (e.g., 2-cyanoethyl); and iPr₂N is diisopropyl amino protecting group. Specific examples of preferred monomers within the scope of Formula III above include but are not limited to polymers defined where X is 2-cyanoethyl, R₁ is C₃ or —(CH₂CH₂O)₃—, and R₂ is DMT or MeNPOC.

As in the case of the protecting groups employed with the linker and nucleotide monomers, the protecting groups employed on the monomers which are used to produce the polyanion chains may be chemically-removable or photolabile protecting groups. Thus, the activator which is employed to remove the photolabile protecting groups is selected from the group consisting of electron beams, gamma rays, x-rays, ultra-violet radiation, light, infra-red radiation, microwaves, electric currents, radiowaves, and combinations thereof. The activator which is employed to remove the chemically-removable protecting groups is selected from the group consisting of acids, bases, oxidants, and reductants. In one preferred embodiment, the protecting groups are photolabile protecting groups and the step of exposing the monomers to an activator comprises applying light to each of the monomers to remove the photolabile protecting group and produce the activated site for reaction with additional monomer. Alternatively, the pre-formed polyanion chains may be attached to each of the protected regions by replacing the chemically-removable protecting group at each of the plurality of protected regions with a photolabile protecting group, and using light-directed methods to attach the polyanion chain to each of the protected regions, or by using any of the other general techniques described above for oligonucleotide synthesis.

In the embodiment wherein the preformed polyanion chain is attached to the desired region(s) of the surface, the method includes attaching to each of the desired region(s) a polymeric polyanion chain of negatively charged phosphate units. One polyanion chain that may be attached to the desired region is a polyanion chain of Formula III-A:

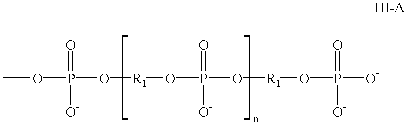

wherein R₁ is selected from the group consisting of a nucleoside moiety, a deoxyribose moiety, C₁₋₈ alkylene, and —(CH₂CH₂O)ₘ— wherein m is an integer from 1 to 8 and n is an integer from 0-18. Other examples of polyanion chains which may be attached to the desired region include but are not limited to polycarboxy cellulose, polycarboxy dextran, polyvinyl phosphoric acid, polyvinyl phosphonic acid, polyglutamate, polyaspartate, and polyacrylic acid.

Suitable pre-formed polyanion chains are advantageously commercially available, or can be produced using techniques known to those skilled in the art.

Attachment of the pre-formed polyanion chain to each of the plurality of designated regions can be accomplished using the same techniques as described above for the attachment of a linker monomer to the designated regions of the surface. Attachment of the pre-formed polyanion chain to each of the plurality of protected regions can be accomplished by exposing the chemically-removable protecting group at each of the protected regions to an activator to provide activated sites and reacting the activated sites with the pre-formed polyanion chain to covalently attach the polyanion chain to each of the protected regions. Alternatively, the pre-formed polyanion chains may be attached to each of the protected regions by replacing the chemically-removable protecting group at each of the plurality of protected regions with a photolabile protecting group, and using light-directed methods to attach the polyanion chain to each of the protected regions.

The formed or attached polyanion chain will include a protecting group at the distal end of the chain (i.e., at the end opposite of the surface of the solid support). In the embodiments wherein the polyanion chain is attached to the designated regions of the surface, the oligonucleotide probes are attached to the distal end of the polyanion chain using the light-directed techniques described above. Specifically, the oligonucleotides can be synthesized on the polyanion chains by exposing the protecting groups on each of the polyanion chains to an activator to remove the protecting group and provide an activated site. This activated site can then be reacted with a first nucleotide monomer to provide the covalent attachment of the first nucleotide monomer to the polyanion chain. In another embodiment, the oligonucleotides are synthesized on the polyanion chains by replacing each of the chemically-removable protecting groups at the distal end of each of the polyanion chains with a photolabile protecting group and attaching a first nucleotide monomer to the distal end of the polyanion chain using light-directed methods, or by using any of the other general techniques described above for oligonucleotide synthesis. In this manner, a first nucleotide monomer is attached to the polyanion chain at each of the plurality of designated regions. Additional nucleotide monomeric units are added to the first nucleotide monomer using the general techniques described above for oligonucleotide synthesis.

As will be apparent to those skilled in the art, the two or more of the foregoing methods of surface modification may be combined to reduce non-specific binding to an oligonucleotide array. For example, the surface of the substrate may be modified by the removal of protecting groups on the protected regions, and attachment of a polyanion chain to the designated regions. As another example, the surface of the substrate may be modified by the replacement of the protecting groups on the protected regions and the attachment of a polyanion chain to the designated regions. As still another example, portions of a region may be treated separately so that, for example, protecting groups are removed from portions of the protected region while polyanion chains are attached at another portion of the protected regions. Many variations of combinations of these techniques will be within the purview of those skilled in the art based upon these examples, and these combinations are thus contemplated by the instant invention.

VII. Oligonucleotide Modification

Other methods for reducing non-specific binding of target molecules to an oligonucleotide array include modifying the oligonucleotides attached to each of the plurality of designated regions on the surface of the solid support. Oligonucleotide modification can be in the form of removing the protecting groups at the distal or terminal end of the oligonucleotide (i.e., the end opposite of the surface to which the oligonucleotide is attached), or replacing the protecting groups with either a negatively charged phosphate residue or a polyanion chain.

Removal of Protecting Groups from the Oligonucleotides

The oligonucleotides produced according to the methods described above have a protecting group at the distal end thereof Each nucleotide monomer which is added to form the oligonucleotide (polymer) has a protecting group which permits the attachment of the next subsequent monomer in the process of building the oligonucleotide. Thus, the last monomer which is added has a protecting group thereon. This protecting group is the protecting group at the distal end of the oligonucleotide.

The protecting group may be removed from the oligonucleotide by exposing each of the oligonucleotides on the surface to an activator to remove the protecting group from each of the plurality of oligonucleotides. The activators listed above for use in oligonucleotide synthesis may be employed for the removal of the protecting groups from the distal end of the oligonucleotide. The removal of the protecting groups from the oligonucleotides is believed to reduce non-specific binding for the reasons already described above.

Replacing Protecting Groups on Oligonucleotides

As an alternative to merely removing the protecting groups from the oligonucleotides, the protecting groups on the oligonucleotides may be replaced with a negatively charged phosphate residue. According to one embodiment, the protecting groups on the oligonucleotides may be replaced with a negatively charged phosphate residue by exposing the plurality of oligonucleotides to an activator to produce activated sites and reacting the activated sites with a compound that covalently bonds a negatively charged phosphate residue to each of the plurality of oligonucleotides. The activator employed may be any of the suitable activators described hereinabove for use in oligonucleotide synthesis and removal of protecting groups. The compounds which are reacted with the activated sites are selected from the group consisting of Formula I:

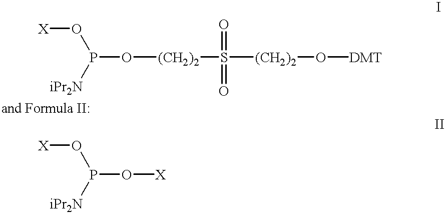

wherein DMT is a dimethoxy trityl protecting group, X is a base-removable protecting group, and iPr₂N is diisopropyl amino protecting group. Examples of suitable compounds of Formulas I and II are provided above.

The reaction of these compounds with the activated site can be carried out by contacting the activated sites on the oligonucleotides to such compounds in the solution phase for a period of time sufficient to effect the covalent attachment of the compounds of Formula I or II to the oligonucleotides. The techniques employed are the same as or analogous to the techniques described above-for oligonucleotide synthesis.

Attachment of Polyanion Chains

In another embodiment, the protecting groups on the oligonucleotides are replaced by attachment of a polyanion chain.

As in the embodiments described above for the attachment of the polyanion chain to the surface of the solid support, the polyanion chain may be attached to the oligonucleotides by either synthesizing or forming the polyanion chain on each of the plurality of oligonucleotides or attaching a pre-formed polyanion chain to each of the oligonucleotides. In the first embodiment, the polyanion chains are formed on the oligonucleotides by attaching a monomer having a negatively charged phosphate unit and a protecting group to each of the oligonucleotides so that a monomer is covalently bound to each of the oligonucleotides. Thereafter, the monomer is exposed to an activator to remove the protecting group, thereby producing activated sites which are capable of reacting with additional monomers to add monomeric units to the first monomer, thereby building the polymer chain. The steps of exposing the monomer to an activator to produced activated sites and reacting with additional monomer are repeated from 0-20 times to produce the polyanion chain on the oligonucleotides. The monomers, activators, and methods employed are as described above for the formation of an oligonucleotide on the surface of the solid support.

In the embodiment wherein the pre-formed polyanion chain is attached to the oligonucleotides, the method includes attaching to each of the desired region(s) a polymeric polyanion chain of negatively charged phosphate units such as the polyanion chains described above. Attachment of the pre-formed polyanion chain to each of the plurality of oligonucleotides can be accomplished using the polymers and techniques as described above for the attachment of a preformed polymer to the surface of the solid support at the protected regions. The formed or attached polyanion chain will include a protecting group at the distal end of the chain (i.e., at the end opposite of the surface of the solid support).

VIII. Combination Techniques

Although utilizing any of the surface modification or oligonucleotide modification techniques described above will reduce non-specific binding of a target molecule to the oligonucleotide array, the techniques described above may also be combined to further reduce non-specific binding.

In one embodiment of the present invention, the protecting groups on the protected regions and the protecting groups on the oligonucleotides are both removed.

In another embodiment, the protecting groups on the protected regions and the protecting groups on the oligonucleotides are both replaced. These protecting groups may be replaced by reaction with a compound of Formula I or II, or by attachment of a polyanion chain. It is also possible to replace the protecting groups on the protected regions of the surface by reaction with a compound of Formula I or II and replace the protecting groups on the oligonucleotides by attachment of a polyanion chain and vice versa. Thus it is not necessary to replace the protecting groups on the protected regions and the oligonucleotides min a consistent manner.

In yet another embodiment, the replacement and removal techniques are combined. For example, the protecting groups on the protected regions of the substrate may be removed while the protecting groups on the oligonucleotides are replaced by either it reaction with a compound of Formula I or II or by attachment of a polyanion chain. Similarly, the protecting groups on the protected regions may be replaced while the protecting groups on the oligonucleotides are removed.

Either of the techniques of removing or replacing protecting groups can also be employed in combination with the method for attaching a polyanion chain to the surface of the solid support. As already noted above, one embodiment of the present invention includes attaching a polyanion chain to either or both of i) each of the designated regions and ii) each of the protected regions on the surface of the solid support. In one embodiment of the present invention, a polyanion chain is attached to each of the plurality of protected regions and the protecting groups on either the polyanion chains attached to the protected regions, or the oligonucleotides, or both are removed. Thus, this embodiment envisions attachment of the polyanion chains to each of the protected regions and removal of the protecting groups on these polyanion chains. This embodiment also envisions attachment of the polyanion chains to each of the protected regions and removal of the protecting groups on the oligonucleotides. Yet another specific example within this embodiment is the array wherein the polyanion chains are attached to each of the protected regions and the protecting groups are removed from both the polyanion chains and the oligonucleotides.

In another embodiment of the invention, a polyanion chain is attached to each of the plurality of protected regions and the protecting groups on either the polyanion chains, or the oligonucleotides, or both are replaced. Replacement of the protecting groups on the polyanion chains and/or the oligonucleotides may be by reaction with a compound of Formula I or II, or by attachment of a polyanion chain, or a combination of these techniques. Attachment of the polyanion chain can be by formation of the polyanion chain or attachment of a pre-formed polyanion chain, as described in detail above. Thus, this embodiment envisions the attachment of a polyanion chain to the protected regions and attachment of a polyanion chain on each of the plurality of oligonucleotides. This embodiment also envisions the attachment of a polyanion chain to the protected regions and replacement of the protecting groups on the oligonucleotides by reaction with a compound of Formula I or II. This embodiment envisions the attachment of a polyanion chain to the protected regions and replacement of the protecting groups on the polyanion chains at the protected regions by reaction with a compound of Formula I or II. Yet another specific example of this embodiment involves the attachment of the polyanion chain to the protected regions and replacement of the protecting groups on the polyanion chains by reaction with a compound of Formula I or II, and also replacement of the protecting groups on the oligonucleotides by attachment of a polyanion chain (either through chain formation or attachment of a pre-formed chain). Although it is not necessary to attach a polyanion chain to the polyanion chains already attached at the protected regions, this embodiment of the present invention is also contemplated. Other specific examples of the many possible modified oligonucleotide arrays contemplated by this embodiment of the invention will be readily apparent to those skilled in the art based upon the preceding description and specific examples.

In another embodiment of the present invention, a polyanion chain is attached at each of the plurality of designated regions and the oligonucleotides are then synthesized on the polyanion chains. In a related embodiment, the polyanion chains are attached at the designated regions and the protecting groups are removed from either the protected regions, or the oligonucleotides (attached to the polyanion chains at the designated regions), or both. Thus, this embodiment envisions attachment of the polyanion chains to each of the designated regions and removal of the protecting groups on the protected regions. This embodiment also envisions attachment of the polyanion chains to each of the designated regions and removal of the protecting groups on the oligonucleotides. Yet another specific example within this embodiment is the array wherein the polyanion chains are attached to each of the designated regions and the protecting groups are removed from both the protected regions and the oligonucleotides.

In another embodiment, the polyanion chains are attached at the designated regions and the protecting groups on either the protected regions, or the oligonucleotides (attached to the polyanion chains at the designated regions), or both are replaced. The replacement of the protecting groups on either or both of the protected regions and the oligonucleotides can be by reaction with a compound of Formula I or II, or attachment of a polyanion chain, or a combination of these techniques. Attachment of the polyanion chain can be by formation of the polyanion chain or attachment of a pre-formed polyanion chain, as described in detail above. Thus, this embodiment envisions the attachment of a polyanion chain to the designated regions and attachment of a second polyanion chain on the oligonucleotide which is in turn attached to the polyanion chain at the designated regions. This embodiment also envisions the attachment of a polyanion chain to the designated regions and replacement of the protecting groups on the protected regions by reaction with a compound of Formula I or II. This embodiment also envisions the attachment of a polyanion chain to the designated regions and replacement of the protecting groups on the oligonucleotides by reaction with a compound of Formula I or II. Yet another specific example of this embodiment involves the attachment of the polyanion chain to the designated regions and replacement of the protecting groups on the protected regions by reaction with a compound of Formula I or II and also replacement of the protecting groups on the oligonucleotides by attachment of a second polyanion chain (either through chain formation or attachment of a pre-formed chain). Other specific examples of the many possible modified oligonucleotide arrays contemplated by this embodiment of the invention will be readily apparent to those skilled in the art based upon the preceding description and specific examples.

In another embodiment of the present invention, a polyanion chain is attached at both i) each of the plurality of designated regions and ii) each of the plurality of protected regions. Thus, in this embodiment, the entire surface of the solid support is coated with the polyanion chains. The oligonucleotides are then synthesized on the polyanion chains attached at each of the designated regions. In a related embodiment, the polyanion chains are attached at both the protected and the designated regions and the protecting groups on either the polyanion chains at the protected regions, or the oligonucleotides (attached to the polyanion chains at the designated regions), or both are removed. Thus, this embodiment envisions attachment of the polyanion chains to each of the protected regions, and each of the designated regions, and removal of the protecting groups on the polyanion chains attached to the protected regions. This embodiment also envisions attachment of the polyanion chains to each of the protected regions and each of the designated regions, and removal of the protecting groups on the oligonucleotides. Yet another specific example within this embodiment is the array wherein the polyanion chains are attached to each of the protected regions and each of the designated regions, and the protecting groups are removed from both the polyanion chains on the protected regions and the oligonucleotides.

In another embodiment, the polyanion chains are attached at both each of the protected regions and each of the designated regions and the protecting groups on either the polyanion chains at the protected regions, or the oligonucleotides (attached to the polyanion chains at the designated regions), or both are replaced. The replacement of the protecting groups on either or both of the polyanion chains at the protected regions and the oligonucleotides can be by reaction with a compound of Formula I or II, or attachment of a polyanion chain, or some combination of these techniques. Attachment of the polyanion chain can be by formation of the polyanion chain or attachment of a pre-formed polyanion chain, as described in detail above. Thus, this embodiment envisions the attachment of a polyanion chain to each of the protected regions and each of the designated regions and attachment of a second polyanion chain on each of the oligonucleotides. This embodiment also envisions the attachment of a polyanion chain to each of the protected regions and each of the designated regions, and replacement of the protecting groups on the polyanion chains at the protected regions by reaction with a compound of Formula I or II. This embodiment also envisions the attachment of a polyanion chain to each of the protected regions and each of the designated regions, and replacement of the protecting groups on the oligonucleotides by reaction with a compound of Formula I or II. Yet another specific example of this embodiment involves the attachment of the polyanion chain to each of the protected regions and each of the designated regions, and replacement of the protecting groups on the polyanion chains at the protected regions by reaction with a compound of Formula I or II and also replacement of the protecting groups on the oligonucleotides by attachment of a second polyanion chain (either through chain formation or attachment of a pre-formed chain). Other specific examples of the many possible modified oligonucleotide arrays contemplated by this embodiment of the invention will be readily apparent to those skilled in the art based upon the preceding description and specific examples.

As should already be apparent to those skilled in the art, the difference between surface modifications involving the step of replacing the protecting groups on the protected regions with a polyanion chain and the attachment of a polyanion chain to those surfaces as a means of derivatizing the support is the timing of each technique. Specifically, the technique of derivatizing the surface of the solid support by attachment of a polyanion chain is carried out prior to synthesizing the oligonucleotide on each of the designated regions. The technique of replacing the protecting groups on the protected region with a polyanion chain is carried out after synthesis of the oligonucleotides on each of the designated regions. This distinction is useful to clarify these techniques and more clearly describe the many possible combinations of techniques which may be employed in carrying out the methods of the present invention. However, as will be apparent to those skilled in the art, the end result will be the same, namely a polyanion chain attached at each of the protected regions on the surface of the solid support.

The following examples are provided to further illustrate the present invention, and should not be construed as limiting thereof. The instant invention is defined solely by the claims which follow.

In the following examples, substrate surfaces were modified to impart a single or multiple negative charge upon completion of the synthesis and final deprotection of a DNA probe array. Experiments were then performed to measure reduction in fluorescent background levels due to surface nonspecific binding NSB of proteins and other components in the sample. The observed background can be of two types: “diffuse” background due to NSB of protein & other soluble components in the sample mixture; and “specular” background due to NSB of insoluble particulate components in the samples, i.e., fine precipitates of metal hydroxides such as may arise from the combination of various buffers used in the sample preparation procedures.

Substrate preparation. Glass wafers (5″×5″×0.027″) for oligonucleotide synthesis were cleaned by soaking successively in Nanostrip (Cyantek, Fremont, Calif.)/15 minutes, 10% aqueous NaOH/70° C./3 minutes, and then 1% aqueous HCl/1 minute, with immediate and thorough rinsing with deionized water between each step. The wafers were then spin-dried for 5 minutes under a stream of nitrogen at 35° C. The slides were then silanated for 15 minutes in a gently agitating solution of N,N-bis(2-hydroxyethyl)-3-aminopropyltriethoxysilane (Gelest, Inc.) (1% vol/vol in 95:5 ethanol-water), rinsed thoroughly with 2-propanol, then deionized water, and finally spin-dried for 5 minutes at 90-110° C.

Array synthesis. Array synthesis was performed on a custom Affymetrix synthesizer, consisting of a mask alignment and UV-exposure system, and a flowcell linked to a reagent delivery system. The substrate was clamped against the flowcell and phosphoramidite addition was carried out according to standard automated oligonucleotide synthesis protocols, specially programmed for delivery of liquid reagents and pressurized argon according to the specific volume and mixing requirements of the flowcell.

### EXAMPLE 1

In this example, the effect of surface modification on nonspecific surface binding of the fluorescent protein conjugate phycoerythrin-streptavidin (“SAPE”) was examined.

A standard, unmodified “control” array was prepared by first adding a hexaethyloxy linker (“HEX”) as the O-MeNPOC-(hexaethyloxy)-O-(2-cyanoethyl)-N,N-diisopropylphosphoramidite (Pease, et al., 1994; McGall, et al., 1997). phosphoramidite, using standard coupling protocols. The substrate was then exposed to UV light through a mask which illuminated alternating square areas of the surface in a checkerboard pattern, and the first MeNPOC-base in the 20-mer sequence (G) was added as a photolabile 5′-O-MeNPOC-(N2-p-isopropylphenoxyacetyl)-2′-deoxyguanosine-3′-O-(2-cyanoethyl)-N,N-diisopropylphosphoramidite (Pease, et al., 1994; McGall, et al., 1997). Alternating cycles of exposure and coupling, using photolabile 5′-O-MeNPOC-nucleoside phosphoramidites, were used to complete the synthesis of the 20 mer oligonucleotide sequence in a checkerboard pattern on the substrate:

“control” array:

background: [substrate]—OPO₂⁽⁻⁾O(CH ₂CH₂O)₆^{-MeNPOC} (unphotolysed)

foreground: [substrate]—O[PO₂⁽⁻⁾O(CH ₂CH₂O)₆]—OPO₂⁽⁻⁾O—

(^{3′} GACTTGCCATCGTAGAACTG^{5′})

The “C3-test” arrays, with surface modification, were prepared as follows: First, a monomeric building block 3-(dimethoxytrityloxy)propyl(2-cyanoethyl)-N,N-diisopropylphosphoramidite (“C3 spacer”, Glen Research, Sterling, VA) was used to add a short oligomer (0, 1, 5) of “C3 spacer” to the substrate using standard coupling/TCA-deprotection protocols. This was optionally followed by the addition of a hexaethyloxy linker (“HEX”), added as a (DMT-hexaethyloxy)phosphoramidite (ChemGenes, Waltham, Mass.), also using standard coupling/TCA-deprotection protocols. The first base (G) of a 20-mer test sequence was then coupled as a photolabile 5′-O-MeNPOC-(N2-p-isopropylphenoxyacetyl)-2′-deoxyguanosine-3′-O-(2-cyanoethyl)-N,N-diisopropylphosphoramidite (Pease, et al., 1994; McGall, et al., 1997). The substrate was then exposed through a mask which illuminated alternate areas of the surface in a checkerboard pattern, and the next MenPOC-base in the 20-mer sequence was added. The alternating cycles of exposure and coupling were used to complete the synthesis of the 20 mer oligonucleotide sequence in a checkerboard pattern on the substrate:

test arrays:

background: [substrate]-O[PO₂⁽⁻⁾O(CH₂)₃O]ₘ(PO₂⁽⁻⁾O(CH₂(CH₂O)₆)ₙOPO₂⁽⁻⁾ O^{3′}—(G)^{5′OH}

foreground: [substrate]-O[PO₂⁽⁻⁾O(CH₂)₃O]ₘ(PO₂⁽⁻⁾O(CH₂(CH₂O)₆)ₙOPO₂ ⁽⁻⁾O^{3′}—(GACTTGCCATCGTAGAACTG)^{5′}

Array hybridization: The control and test arrays were then hybridized with a solution of the complementary 5′-biotinylated target oligonucleotide (^{5′}CTGAACGGTAGCATCTTGAC^{3′}) and the bound oligonucleotide target was then “stained” with a solution of phycoerythrin-streptavidin conjugate (“SAPE”, Molecular Probes, Inc.) using the following standard GeneChip™ protocols on a Affymetrix hybridization station:

HYBWASH A conditions:

Concentration of oligo=100 pM in 5×SSPE/0.05% triton X-100

Volume of oligo in sample vial=650 ml

Wash buffer=6×SSPE/0.005% triton X-100

Hyb temperature=50° C.

Hyb time=1800 seconds

Wash temperature=50° C.

Number of wash cycles=4

Number of drain & fills per cycle=4

Hold temperature=20° C.

Staining conditions:

SAPE=1:500 dilution in 6×SSPE/0.005% triton X-100

Volume of SAPE in wash vial=200 ml

Stain temperature=RT

Rotisserie speed=60 rpm

Duration=5 minutes

WASHA conditions:

Wash temperature=22° C.

Number of wash cycles=10

Number of drain & fills per cycle=2

Hold temperature=20° C.

Array scanning: Surface fluorescence levels were extracted from images acquired on a Hewlett-Packard GeneArray™ confocal fluorescence scanner:

Scan conditions:

Scan temperature=20° C.

Pixel size=30 m

Filter=560 nm

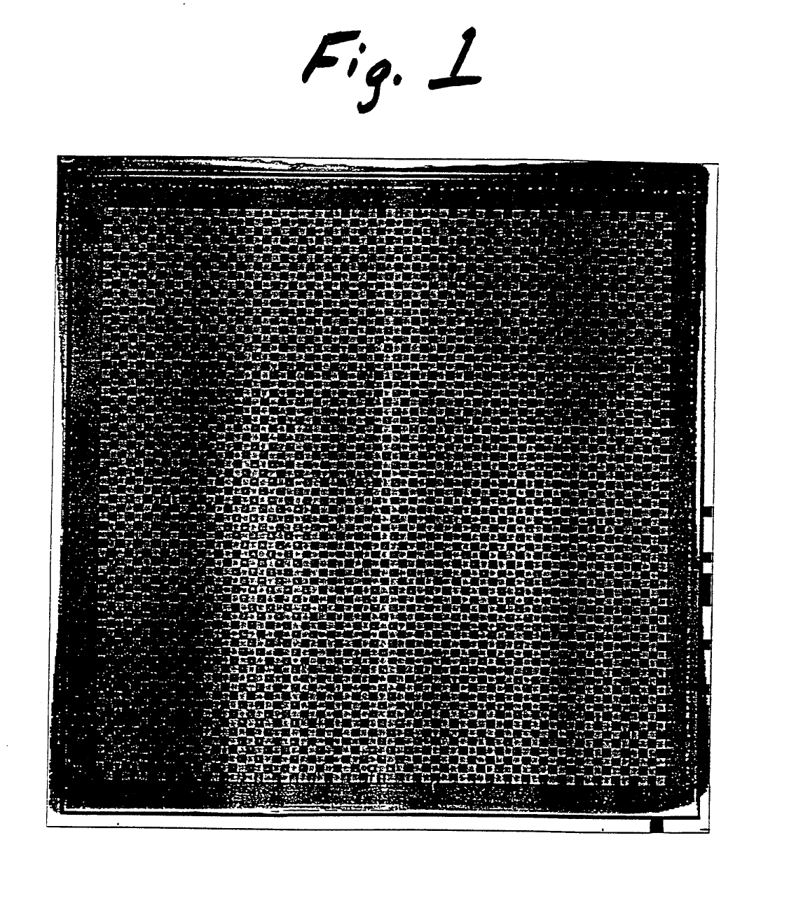

A typical image is displayed in FIG. 1, and the data is summarized in the Table 1 below. Measured “foreground” fluorescence intensities correspond to signal intensity within squares of the checkerboard array containing the oligonucleotide probe sequence (“+”), and “background” fluorescence was determined within squares of the checkerboard array which did not contain probe sequence (“−”) It can be seen that, compared to the control array, modification of the substrate with at least one “C3” prior to array synthesis resulted in a six-fold reduction in background fluorescence intensity.

TABLE 1

| | | foreground | background | |
|---|---|---|---|---|
| | | fluorescence | fluorescence | |
| surface treatment: | | intensity (S) | intensity (N) | S / N |
| control | | 9650 | 348 | 28 |
| (HEX only) | | | | |
| m = | 1 | 10780 | 61 | 176 |
| n = | 0 | | | |
| m = | 1 | 13010 | 56 | 232 |
| n = | 1 | | | |
| m = | 5 | 10525 | 53 | 198 |
| n = | 0 | | | |
| m = | 5 | 20035 | 55 | 364 |
| n = | 5 | | | |

### EXAMPLE 2

This example was aimed at examining the effect of surface modifications on both diffuse and specular NSB. The latter is frequently observed in the more complex samples typically analysed with GeneChip arrays.

The “control” array was prepared exactly as described for the control arrays in Example 1:

background: [substrate]—OPO₂⁽⁻⁾O(CH₂CH₂O)₆^{—O-MeNPOC} (non-photolysed)

foreground: [substrate]—O[PO₂⁽⁻⁾O(CH₂CH₂O)₆]—OPO₂⁽⁻⁾O—

(^{3′}GACTTGCCATCGTAGAACTG^{5′})

A “control with photolysis” array was prepared in the same way as the control, except that after oligonucleotide synthesis, the entire array was exposed to light to remove the remaining MeNPOC photolabile groups from non-synthesized (“−”) regions of the HEX-modified substrate:

background: [substrate]—OPO₂⁽⁻⁾O(CH₂CH₂O)₆^{—OH} (photolysed)

foreground: [substrate]—O[PO₂⁽⁻⁾O(CH₂CH₂O)₆]—OPO₂⁽⁻⁾O—

(^{3′}GACTTGCCATCGTAGAACTG^{5′OH}

The “C3-test” arrays, with (C3)ₙ(HEX) surface modification, were prepared according to the method described for the “C3-test” arrays in Example 1:

background: [substrate]-O[PO₂⁽⁻⁾O(CH₂)₃O]ₘ(PO₂^{(-)O(CH}₂CH₂O)₆)—OPO₂⁽⁻⁾ O^{3′}—(G)^{5′OH}

foreground: [substrate]-O[PO₂⁽⁻⁾O(CH₂)₃O]ₘ(PO₂⁽⁻⁾O(CH₂CH₂O)₆)—OPO₂^{(-)O—}^{3′}(GACTTGCCATCGTAGAACTG)^{5′OH′}

Hybridization: Arrays were hybridized with a solution of the complementary 5′-biotinylated target oligonucleotide (^{5′}CTGAACGGTAGCATCTTGAC^{3′}) spiked into a GeneChip test sample at 100 pM final concentration. The GeneChip test sample was prepared according to protocols provided with the Affymetrix P450 assay kit package insert. It is essentially prepared from an unpurified multiplex PCR reaction, which is fragmented with DNAse-I, and labelled with biotin-dideoxyATP in the presence of terminal transferase. In some cases, 1 mg/ml of bovine serum albumen (BSA) was also included in the sample to examine it's ability to provide further reduction in the NSB. Hybridization, SAPE staining, washing and scanning were performed as described in Example 1 above.

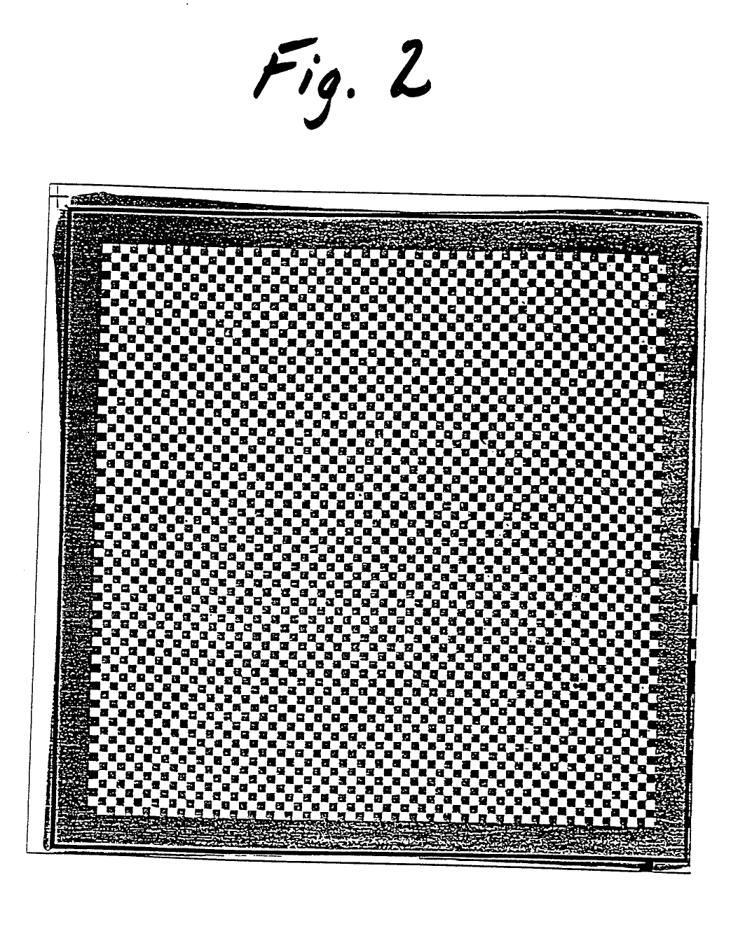

Typical images are displayed in FIGS. 2 and 3, and the data is summarized in Table 2 below. FIG. 2 corresponds to the data in entry 1 of Table 2. FIG. 3 corresponds to the data in entry 3 of Table 2. Measured “foreground” fluorescence intensities correspond to signal intensity within squares of the checkerboard array containing the oligonucleotide probe sequence (“+”), and “background” fluorescence was determined within squares of the checkerboard array which did not contain probe sequence (“−”). A number of conclusions can be made from this data. Firstly, in the case of the control array with HEX-modified substrate (no C3 modification), final photolytic removal of MeNPOC protecting groups from non-probe containing regions caused a significant reduction in the diffuse NSB levels in those areas (entries 1 & 2). Photolysis also reduced, but did not eliminate, the rather severe specular background that was observed on the non-photolysed control array. Modification of the substrate with “C3” oligomers (length 5-20) prior to HEX addition and oligonucleotide synthesis resulted in substantial reductions in background fluorescence intensity, and almost completely eliminated specular background. Inclusion of BSA in samples appeared to provide the lowest diffluse background levels.

TABLE 2

| | | | fore- | back- | | relative |
|---|---|---|---|---|---|---|
| | | | ground | ground | | specular |
| | | | fluores- | fluores- | | back- |
| | | | cence | cence | | ground |
| | substrate | +/− | intensity | intensity | | fluores- |
| entry | treatment | BSA | (S) | (N) | S / N | cence |
| 1 | “control”: | − | 3750 | 650 | 5.8 | HIGH |
| | no C3 (n = 0); − | | | | | |
| | substrate | | | | | |
| | photolysis | | | | | |
| 2 | no C3 (n = 0); + | − | 5700 | 900 | 6.3 | ME- |
| | substrate | | | | | DIUM- |
| | photolysis | | | | | LOW |
| 3 | + C3 (n = 5) | + | 3360 | 170 | 20 | LOW |
| 4 | + C3 (n = 10) | − | 3110 | 220 | 14 | LOW |
| 5 | + C3 (n = 15) | + | 3105 | 120 | 26 | LOW |
| 6 | + C3 (n = 20) | − | 4600 | 150 | 31 | LOW |

The foregoing is illustrative of the present invention and is not to be construed as limiting thereof. The invention is defined by the following claims, with equivalents of the claims to be included therein.

That which is claimed is:

1. A method for reducing non-specific binding of a target molecule to a plurality of oligonucleotides on a surface of a solid support, wherein said surface has a plurality of designated regions and a plurality of protected regions, each of said plurality of protected regions having a protecting group thereon, said method comprising:
a) producing said plurality of oligonucleotides at each of said designated regions, each of said plurality of oligonucleotides having a terminal protecting group; and
b) replacing with a negatively charged phosphate residue, at least one of:
i) the protecting groups on each of said plurality of oligonucleotides produced in step a), and
ii) the protecting groups on each of said plurality of protected regions; whereby non-specific binding of said target molecule is reduced.

2. The method according to claim 1, wherein said solid support comprises polymerized Langmuir Blodgett film, functionalized glass, germanium, silicon, polymers, (poly)tetrafluoroethylene, polystyrene, gallium arsenide, metal oxide films, and combinations thereof.

3. The method according to claim 1, wherein said step a) of producing said plurality of oligonucleotides comprises:
1) attaching to each of said designated regions an independently selected linker monomer having a photolabile protecting group;
2) attaching an independently selected nucleotide monomer having a photolabile protecting group to each of said attached linker monomers using light directed methods to produce a plurality of oligonucleotides having a terminal photolabile protecting group; and
3) repeating step 2) from 1 to 120 times, to attach subsequent nucleotide monomers to each of said oligonucleotides produced in step 2) to produce a plurality of oligonucleotides having a terminal photolabile protecting group.

4. The method according to claim 1, wherein said step a) of producing said plurality of oligonucleotides comprises:
1) attaching to each of said designated regions an independently selected linker monomer having a chemically-removable protecting group;
2) replacing each of said chemically-removable protecting groups on each of said attached linker monomers with a photolabile protecting group;
3) attaching an independently selected nucleotide monomer having a chemically-removable protecting group to each of said attached linker monomers using light-directed methods to produce a plurality of oligonucleotides having a terminal chemically-removable protecting group;
4) replacing each of said chemically-removable protecting groups on each of said oligonucleotides with a photolabile protecting group; and
5) repeating steps 3) and 4) from 1 to 120 times, to attach subsequent nucleotide monomers to each of said oligonucleotides produced in step 3) to produce said plurality of oligonucleotides having a terminal chemically-removable protecting group.

5. The method according to claim 1, wherein said step a) of producing said plurality of oligonucleotides comprises:
1) attaching to each of said designated regions an independently selected linker monomer having a chemically-removable protecting group;
2) forming an activation layer on said designated regions and said protected regions, said activation layer comprising:
i) a photoactive agent, said photoactive agent producing a catalyst when irradiated, and
ii) an autocatalytic agent, said autocatalytic agent generating a product that removes said chemically-removable protecting group when said autocatalytic agent is activated by said catalyst;
3) irradiating a portion of said activation layer overlying said designated regions to remove said chemically-removable protecting group on said linker monomer;
4) attaching an independently selected nucleotide monomer having a chemically-removable protecting group to each of said attached linker monomers, to produce a plurality of oligonucleotides having a terminal chemically-removable protecting group;
5) irradiating a portion of said activation layer overlying said designated regions to remove said chemically-removable protecting group on said oligonucleotides;
6) repeating steps 4) and 5) from 1 to 120 times, to attach subsequent nucleotide monomers to each of said oligonucleotides produced in step 4) to produce said plurality of oligonucleotides having a terminal chemically-removable protecting group.

6. The method according to claim 1, wherein said step b) of replacing comprises:
1) exposing at least one of: i) each of said plurality of oligonucleotides and ii) each of said plurality of protected regions, to an activator to remove the protecting groups, to produce activated sites; and
2) reacting said activated sites with a compound that covalently bonds a negatively charged phosphate residue to at least one of i) each of said plurality of oligonucleotides and ii) each of said plurality of protected regions.

7. The method according to claim 6, wherein said protecting group is a photolabile protecting group and said activator is selected from the group consisting of ion beams, electron beams, gamma rays, x-rays, ultra-violet radiation, light, infra-red radiation, microwaves, electric currents, radiowaves, and combinations thereof.

8. The method according to claim 6, wherein said protecting group is a chemically-removable protecting group and said activator is selected from the group consisting of acids, bases, oxidants, and reductants.

9. The method according to claim 6, wherein said step 2) of reacting said activated sites with a compound comprises reacting each of said activated sites with a compound selected from the group consisting of Formula I: 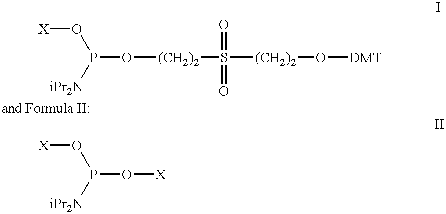
wherein:
DMT is a dimethoxy trityl protecting group;
each X is a base-removable protecting group; and
iPr₂N is diisopropyl amino protecting group.

10. The method according to claim 1, wherein said step b) of replacing comprises:
1) exposing at least one of: i) each of said plurality of oligonucleotides and ii) each of said plurality of protected regions, to an activator to remove the protecting groups to produce activated sites;
2) reacting said activated sites with a monomer having a negatively charged phosphate unit and a protecting group, whereby said monomer is covalently bound to at least one of i) each of said plurality of oligonucleotides and ii) each of said plurality of protected regions; and
3) repeating steps 1) and 2) from 1 to 20 times to produce a polyanion chain of negatively charged phosphate units on at least one of i) each of said plurality of oligonucleotides and ii) each of said plurality of protected regions.

11. The method according to claim 10, wherein said step 2) comprises reacting with a monomer of Formula III: 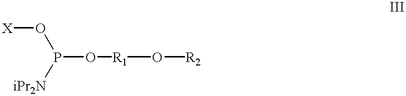
wherein:
R₁ is selected from the group consisting of a nucleoside moiety, a deoxyribose moiety, C₁₋₈ alkylene, and —(CH₂CH₂O)ₘ— wherein m is an integer from 1 to 8;
R₂ is a protecting group selected from the group consisting of a dimethoxy trityl protecting group and a MeNPOC protecting group;
X is a base-removable protecting group; and
iPr₂N is diisopropyl amino protecting group.

12. The method according to claim 1, wherein said step b) of replacing comprises replacing both i) the protecting groups on each of said plurality of oligonucleotides produced in step a), and ii) the protecting groups on each of said plurality of protected regions.

13. A method for reducing non-specific binding of a target molecule to a plurality of oligonucleotides on a surface of a solid support, wherein said surface has a plurality of designated regions and a plurality of protected regions, each of said plurality of protected regions having a protecting group thereon, said method comprising:
a) attaching a polyanion chain having a protecting group to each of said designated regions;
b) forming said plurality of oligonucleotides on each of said polyanion chains at each of said designated regions, each of said plurality of oligonucleotides having a terminal protecting group;
whereby non-specific binding of said target molecule is reduced.

14. The method according to claim 13, wherein said solid support comprises polymerized Langmuir Blodgett film, functionalized glass, germanium, silicon, polymers, (poly)tetrafluoroethylene, polystyrene, gallium arsenide, metal oxide films, and combinations thereof.

15. The method according to claim 13, wherein said step a) of attaching a polyanion chain comprises forming said polyanion chain on each of said protected regions, wherein said step of forming said polyanion chain comprises:
1) attaching a monomer to each of said designated regions, said monomer having a negatively charged phosphate unit and a protecting group, whereby said monomer is covalently bound to each of said designated regions;
2) exposing each of said monomers to an activator to remove the protecting group from each of said monomers to produce activated sites;
3) reacting said activated sites with said monomer having a negatively charged phosphate unit and a protecting group, to add a second monomer; and
4) repeating steps 2) and 3) from 0 to 20 times to produce a polyanion chain having a protecting group on each of said designated regions.

16. The method according to claim 15, wherein said steps 1) and 3) comprise using a monomer of Formula III: 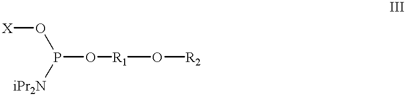
wherein:
R₁ is selected from the group consisting of a nucleoside moiety, a deoxyribose moiety, C₁₋₈ alkylene, and —(CH₂CH₂O)ₘ— wherein m is an integer from 1 to 8;
R₂ is selected from the group consisting of a dimethoxy trityl protecting group and a MeNPOC protecting group;
X is a base-removable protecting group; and
iPr₂N is diisopropyl amino protecting group.

17. The method according to claim 15, wherein said protecting groups are photolabile protecting groups and said step 2) comprises using an activator selected from the group consisting of ion beams, electron beams, gamma rays, x-rays, ultra-violet radiation, light, infra-red radiation, microwaves, electric currents, radiowaves, and combinations thereof.

18. The method according to claim 15, wherein said protecting groups are chemically-removable protecting groups and said step 2) comprises using an activator selected from the group consisting of acids, bases, oxidants, and reductants.

19. The method according to claim 13, wherein said step a) of attaching a polyanion chain further comprises attaching a polyanion chain having a protecting group to each of said protected regions.

20. The method according to claim 13, wherein said protecting groups comprise photolabile protecting groups and said step b) of forming said plurality of oligonucleotides comprises:
1) attaching an independently selected nucleotide monomer having a terminal photolabile protecting group to each of said polyanion chains at each of said designated regions using light directed methods;
2) repeating step 1) from 1 to 120 times, to attach subsequent nucleotide monomers to each of said nucleotide monomers attached in step 1) to produce a plurality of oligonucleotides having a terminal photolabile protecting group.

21. The method according to claim 13, wherein said protecting groups comprise chemically-removable protecting groups and said step b) of forming said plurality of oligonucleotides comprises:
1) replacing each of said chemically-removable protecting groups on each of said attached polyanion chains at each of said designated regions with a photolabile protecting group;
2) attaching an independently selected nucleotide monomer having a chemically-removable protecting group to each of said attached polyanion chains using light-directed methods to produce a plurality of attached nucleotide monomers having a chemically-removable protecting group;
3) replacing each of said chemically-removable protecting groups on each of said nucleotide monomers with a photolabile protecting group;
4) attaching an independently selected nucleotide monomer having a chemically-removable protecting group to each of said attached nucleotide monomers using light directed methods to produce a plurality of oligonucleotides having a terminal chemically-removable protecting group;
5) repeating steps 3) and 4) from 1 to 120 times, to attach subsequent nucleotide monomers to each of said oligonucleotides produced in step 3) to produce said plurality of oligonucleotides having a terminal chemically-removable protecting group.

22. The method according to claim 13, wherein said protecting groups comprise chemically-removable protecting groups and said step a) of producing said plurality of oligonucleotides comprises:
1) forming an activation layer over said designated regions and said protected regions, said activation layer comprising:
i) a photoactive agent, said photoactive agent producing a catalyst when irradiated, and
ii) an autocatalytic agent, said autocatalytic agent generating a product that removes said chemically-removable protecting group when said autocatalytic agent is activated by said catalyst;
2) irradiating a portion of said activation layer overlying said designated regions to remove said chemically-removable protecting group on said polyanion chain;
3) attaching an independently selected nucleotide monomer to each of said attached polyanion chains at each of said designated regions, to produce a plurality of nucleotide monomers having a terminal chemically-removable protecting group;
4) irradiating a portion of said activation layer overlying said designated regions to remove said chemically-removable protecting group on each of said nucleotide monomers;
5) repeating steps 3) and 4) from 1 to 120 times, to attach subsequent nucleotide monomers to each of said nucleotides attached in step 3) to produce said plurality of oligonucleotides having a terminal chemically-removable protecting group.

23. The method according to claim 13, wherein said step a) of attaching a polyanion chain further comprises attaching a polyanion chain having a protecting group to each of said protected regions.

24. A method for reducing non-specific binding of a target molecule to a plurality of oligonucleotides on a surface of a solid support, wherein said surface has a plurality of designated regions and a plurality of protected regions, each of said plurality of protected regions having a protecting group thereon, said method comprising:
a) attaching a polyanion chain having a protecting group to each of said protected regions; and
b) forming said plurality of oligonucleotides at each of said designated regions, each of said plurality of oligonucleotides having a terminal protecting group; whereby non-specific binding of said target molecule is reduced.

25. A method for reducing non-specific binding of a target molecule to a plurality of oligonucleotides on a surface of a solid support, wherein said surface has a plurality of designated regions and a plurality of protected regions, each of said plurality of protected regions having a protecting group thereon, said method comprising:
a) attaching a polyanion chain having a protecting group to each of said designated regions;
b) forming said plurality of oligonucleotides on said polyanion chains at each of said designated regions, each of said plurality of oligonucleotides having a terminal protecting group; and
c) removing at least one of:
i) the protecting groups on each of said plurality of oligonucleotides produced in step b), and
ii) the protecting groups on each said plurality of protected regions; whereby non-specific binding of said target molecule is reduced.

26. The method according to claim 25, wherein said step c) of removing comprises removing both i) the protecting groups on each of said plurality of oligonucleotides produced in step b), and ii) the protecting groups on each of said plurality of protected regions.

27. The method according to claim 25, wherein said step a) of attaching a polyanion chain further comprises attaching a polyanion chain having a protecting group to each of said protected regions.

28. The method according to claim 27, wherein said step c) of removing comprises removing both i) the protecting groups on each of said plurality of oligonucleotides produced in step b), and ii) the protecting groups on each of said plurality of polyanion chains attached to each of said protected regions.

29. A method for reducing non-specific binding of a target molecule to a plurality of oligonucleotides on a surface of a solid support, wherein said surface has a plurality of designated regions and a plurality of protected regions, each of said plurality of protected regions having a protecting group thereon, said method comprising:
a) attaching a polyanion chain having a protecting group to each of said protected regions;
b) forming said plurality of oligonucleotides at each of said designated regions, each of said plurality of oligonucleotides having a terminal protecting group; and
c) removing at least one of:
i) the protecting groups on each of said plurality of oligonucleotides produced in step b), and
ii) the protecting groups on each said plurality of polyanion chains attached to each of said protected regions;
whereby non-specific binding of said target molecule is reduced.

30. The method according to claim 29, wherein said step c) of removing comprises removing both i) the protecting groups on each of said plurality of oligonucleotides produced in step b), and ii) the protecting groups on each of said plurality of polyanion chains attached to each of said protected regions.

31. A method for reducing non-specific binding of a target molecule to a plurality of oligonucleotides on a surface of a solid support, wherein said surface has a plurality of designated regions and a plurality of protected regions, each of said plurality of protected regions having a protecting group thereon, said method comprising:
a) attaching a polyanion chain having a protecting group to each of said designated regions;
b) forming said plurality of oligonucleotides on said polyanion chains at each of said designated regions, each of said plurality of oligonucleotides having a terminal protecting group; and
c) replacing with a negatively charged phosphate residue, at least one of:
i) the protecting groups on each of said plurality of oligonucleotides produced in step b), and
ii) the protecting groups on each said plurality of protected regions;
whereby non-specific binding of said target molecule is reduced.

32. The method according to claim 31, wherein said step c) of replacing comprises:
1) exposing at least one of: i) each of said plurality of oligonucleotides and ii) each of said plurality of protected regions, to an activator to remove the protecting groups to produce activated sites; and
2) reacting said activated sites with a compound that covalently bonds a negatively charged phosphate residue to at least one of i) each of said plurality of oligonucleotides and ii) each of said plurality of protected regions.

33. The method according to claim 31, wherein said step c) of replacing comprises:
1) exposing at least one of: i) each of said plurality of oligonucleotides and ii) each of said plurality of protected regions, to an activator to remove the protecting groups, to produce activated sites;
2) reacting said activated sites with a monomer having a negatively charged phosphate unit and a protecting group, whereby said monomer is covalently bound to at least one of: i) each of said plurality of oligonucleotides and ii) each of said plurality of protected regions; and
3) repeating steps 1) and 2) from 1 to 20 times to produce a polyanion chain of negatively charged phosphate units on at least one of: i) each of said plurality of oligonucleotides and ii) each of said plurality of protected regions.

34. The method according to claim 31, wherein said step c) of replacing comprises replacing both i) the protecting groups on each of said plurality of oligonucleotides produced in step b), and ii) the protecting groups on each of said plurality of protected regions, with a negatively charged phosphate residue.

35. The method according to claim 31, wherein said step a) of attaching a polyanion chain further comprises attaching a polyanion chain having a protecting group to each of said protected regions.

36. The method according to claim 35, wherein said step c) of replacing comprises replacing both i) said protecting groups on each of said plurality of oligonucleotides produced in step b), and ii) said protecting groups on each said plurality of polyanion chains attached to each of said protected regions, with a negatively charged phosphate residue.

37. A method for reducing non-specific binding of a target molecule to a plurality of oligonucleotides on a surface of a solid support, wherein said surface has a plurality of designated regions and a plurality of protected regions, each of said plurality of protected regions having a protecting group thereon, said method comprising:
a) attaching a polyanion chain having a protecting group to each of said protected regions;
b) forming said plurality of oligonucleotides at each of said designated regions, each of said plurality of oligonucleotides having a terminal protecting group; and
c) replacing with a negatively charged phosphate residue, at least one of:
i) the protecting groups on each of said plurality of oligonucleotides produced in step b), and
ii) the protecting groups on each said plurality of polyanion chains attached to each of said protected regions;
whereby non-specific binding of said target molecule is reduced.

38. The method according to claim 37, wherein said step c) of replacing comprises replacing both i) said protecting groups on each of said plurality of oligonucleotides produced in step b), and ii) said protecting groups on each said plurality of polyanion chains attached to each of said protected regions, with a negatively charged phosphate residue.

39. A solid support for solid-phase synthesis comprising a surface having a plurality of designated regions and a plurality of protected regions, wherein a polyanion chain having a protecting group is attached to at least one of: i) each of said designated regions, and ii) each of said protected regions.

40. A solid support according to claim 39, wherein a polyanion chain having a protecting group is attached to both i) each of said designated regions, and ii) each of said protected regions.

41. A method for screening a target molecule for hybridization to a plurality of oligonucleotides, said method comprising:
a) providing a solid support comprising a surface having a plurality of designated regions and a plurality of protected regions, wherein a polyanion chain having a protecting group is attached to at least one of: i) each of said designated regions, and ii) each of said protected regions, and wherein one of said plurality of oligonucleotides is at each of said plurality of designated regions, said oligonucleotide being attached to said polyanion chain when said polyanion chain is attached to each of said designated regions;
b) contacting said target molecule to said plurality of oligonucleotides; and
c) detecting hybridization of said target molecule to said plurality of oligonucleotides;
wherein said polyanion chain reduces non-specific binding of said target molecule to said plurality of oligonucleotides.

42. The method according to claim 41, wherein said polyanion chain is attached to both i) each of said designated regions, and ii) each of said protected regions, and wherein each of said plurality of oligonucleotides is attached to said polyanion chain at each of said designated regions.
